(19) United States
(12) Patent Application Publication
(10) Pub. No.: US 2024/0164837 A1
(43) Pub. Date: May 23, 2024

(54) IMAGE-GUIDED ANNULOPLASTY

(71) Applicant: Navix International Limited, Road Town, Tortola (VG)

(72) Inventor: Shlomo BEN-HAIM, Milan (IT)

(73) Assignee: Navix International Limited, Road Town, Tortola (VG)

(21) Appl. No.: 17/772,680

(22) PCT Filed: Oct. 29, 2020
(86) PCT No.: PCT/IB2020/060167

§ 371 Date: Apr. 28, 2022

Related U.S. Application Data
(60) Provisional application No. 62/978,894, filed on Feb. 20, 2020.
(60) Provisional application No. 62/927,712, filed on Oct. 30, 2019.

(51) Int. Cl. A61B 34/10

(52) U.S. Cl. CPC A61B 34/10; A61B 2034/105

(57) ABSTRACT

Methods and devices using measurements of heart electrophysiological activity to guide structural heart disease interventions, and in some particular embodiments, implantation of heart valve annuloplasty devices. In some embodiments, measurements of heart electrophysiological activity are mapped into locations of a heart model defined by one or more additional measurement modalities. Locations to map electrophysiological data to, in some embodiments, are determined by non-electrophysiological measurements simultaneous with the electrophysiological data measurement which locate a probe—for example, measurements made by the probe itself, and/or measurements which themselves indicate positioning of the probe.

### RELATED APPLICATIONS

This application claims the benefit of priority under 35 USC § 119) of U.S. Provisional Patent Application No. 62/927,712 filed Oct. 30, 2019 and U.S. Provisional Patent Application No. 62/978,894 filed Feb. 20, 2020.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

### SUMMARY OF THE INVENTION

According to an aspect of some embodiments of the present disclosure, there is provided a method of mapping a vascular lumen extending alongside a body cavity, the method including: moving a first probe within the body cavity while measuring signals from a plurality of electrical fields; moving a second probe through the vascular lumen while measuring signals from the plurality of electrical fields; reconstructing a shape of the body cavity and the vascular lumen using the measurements of the first probe and the second probe; and identifying vascular lumen positions within the reconstructed shape corresponding to measurement positions of the second probe.

According to some embodiments of the present disclosure, the method includes displaying the reconstructed shape with an indication of the vascular lumen positions.

According to some embodiments of the present disclosure, the body cavity includes one or more heart chambers, and the vascular lumen includes coronary vasculature.

According to some embodiments of the present disclosure, the method includes tracking the position of a potentially traumatizing device as it moves through the body cavity, and estimating the distance of the potentially traumatizing device to the vascular lumen positions.

According to some embodiments of the present disclosure, the potentially traumatizing device is a fastener for an implantable device.

According to some embodiments of the present disclosure, the implantable device is an annuloplasty device.

According to some embodiments of the present disclosure, the method includes providing an indication that the potentially traumatizing device is within a potentially hazardous proximity to the vascular lumen.

According to an aspect of some embodiments of the present disclosure, there is provided a method of locating a heart valve annulus along an atrioventricular axis, the method including: measuring intracardiac electrophysiological signal waveforms from probe positions extending between an atrial side and a ventricular side of a heart valve annulus, including, for each side, a respective plurality of probe positions; determining relative spatial locations of the probe positions within the heart; and identifying, based on the signal waveform measurements, a position and orientation of a region between the atrial side and the ventricular side, the region being positioned at the heart valve annulus along the atrioventricular axis, and oriented to include opposite circumferential sides of the heart valve annulus.

According to some embodiments of the present disclosure, the spatial locations correspond to locations within a 3-D model of the heart.

According to some embodiments of the present disclosure, the method includes displaying the 3-D model of the heart marked with the identified spatial locations at the position of the heart valve annulus along the atrioventricular axis.

According to some embodiments of the present disclosure, the method includes identifying at least one of the spatial locations as being at the position of an atrium along the atrioventricular axis, based on the signal waveform measured at the at least one of the spatial locations.

According to some embodiments of the present disclosure, the method includes displaying a 3-D model of the heart marked with the identified at least one of the spatial locations at the position of the atrium along the atrioventricular axis.

According to some embodiments of the present disclosure, the method includes identifying at least one of the spatial locations as being at the position of a ventricle along the atrioventricular axis, based on the signal waveform measured at the at least one of the spatial locations.

According to some embodiments of the present disclosure, the method includes displaying a 3-D model of the heart marked with the identified at least one of the spatial locations at the position of the ventricle along the atrioventricular axis.

According to some embodiments of the present disclosure, the identification of position along the atrioventricular axis includes identification of relative amplitudes of an atrially-generated electrophysiological signal, and a ventricularly-generated electrophysiological signal.

According to some embodiments of the present disclosure, the atrially generated signal includes a P wave of an electrocardiogram.

According to some embodiments of the present disclosure, the ventricularly generated signal includes a QRS complex of an electrocardiogram.

According to some embodiments of the present disclosure, the identifying includes interpolating between atrial-side and ventricular side measurements of the electrophysiological signal waveforms to identify one or more intermediate positions at the heart valve annulus.

According to some embodiments of the present disclosure, the identifying identifies a planar region intersecting an entire circumference of the valve annulus.

According to some embodiments of the present disclosure, the identifying identifies a non-planar region intersecting an entire circumference of the valve annulus.

According to some embodiments of the present disclosure, the non-planar region is saddle-shaped due to a geometric deformity of the valve annulus.

According to an aspect of some embodiments of the present disclosure, there is provided a method of automatically locating a hinge of a heart valve annulus, the method including: accessing a 3-D model including the heart valve annulus and at least a portion of the heart valve leaflets; determining elevation angles of surface orientation relative to the valve annulus along a plurality of radii of the valve annulus; and identifying, along the plurality of radii, respective positions that are portions of the hinge, based on elevation angle and/or changes in elevation angle along its respective radius.

According to some embodiments of the present disclosure, the location of the heart valve annulus surface used in determining the elevation angles is at least partially determined based on electrophysiological measurements measured from positions along an atrioventricular axis extending through the heart valve annulus.

According to an aspect of some embodiments of the present disclosure, there is provided a method of identifying extents of fibrous tissue within a heart valve annulus, the method including: measuring an electrical signal indicating impedance changes from probe positions in proximity to tissue extending from connective tissue of a valve annulus to myocardial tissue surrounding the valve annulus; determining relative spatial locations of the probe positions within the heart; and identifying a plurality of the spatial locations as including connective tissue of the valve annulus, based on the measured electrical signal.

According to some embodiments of the present disclosure, the method includes displaying an indication of the identification.

According to some embodiments of the present disclosure, the method includes measuring a time course of changes in the measured electrical signal due to motion of tissue, and identifying a plurality of the spatial locations as including connective tissue of the valve annulus, distinct from leaflets of the valve, based on the measured time course of changes in the electrical signal.

According to an aspect of some embodiments of the present disclosure, there is provided a method of detecting valve leaflets, the method including: measuring electrical signals indicative of impedance changes from one or more electrodes located near a cardiac valve; and determining if the electrode is near a leaflet of the cardiac valve, based on a characteristic of electrical signal changes during a single heartbeat cycle.

According to an aspect of some embodiments of the present disclosure, there is provided a method of identifying hinge boundary between a heart valve annulus and heart valve leaflets, the method including: measuring, at a plurality of probe positions in proximity to one or both of the heart valve annulus and the heart valve leaflets, a time course of changes in an electrical signal indicative of impedance changes due to motion of tissue; determining relative spatial locations of the probe positions within the heart; analyzing motions detected at the plurality of probe positions as characteristic of motions of the valve annulus or of the valve leaflets; and identifying as valve hinge positions spatial locations between valve annulus locations and valve leaflet locations.

According to some embodiments of the present disclosure, the method includes presenting an indication of the identifications of valve hinge positions.

According to some embodiments of the present disclosure, motion-induced electrical signal changes characteristic of the valve leaflets are indicative of a doubled cycle of increasing and decreasing impedance during a single heartbeat cycle.

According to some embodiments of the present disclosure, motion-induced electrical signal changes characteristic of the valve annulus are indicative of a single cycle of increasing and decreasing impedance during a single heartbeat cycle.

According to some embodiments of the present disclosure, the location of the heart valve annulus along the atrioventricular axis is determined based on electrophysiological measurements measured from positions along the atrioventricular axis; and the probe positions selected according to the determined location of the heart valve annulus.

According to an aspect of some embodiments of the present disclosure, there is provided a method of locating a structure of the electrical conduction system of the heart, the method including: measuring intracardiac electrophysiological signal waveforms from intracardial probe positions; determining spatial locations of the intracardial probe positions within the heart; and identifying at least one of the spatial locations as being at the position of the structure, based on the signal waveform measured at the at least one of the spatial locations.

According to some embodiments of the present disclosure, the structure includes a bundle of His, and the signal waveform is characteristic of a latency to waveform arrival at the bundle of His.

According to some embodiments of the present disclosure, the structure includes an AV node, and the signal waveform is characteristic of a latency to waveform arrival at the AV node.

According to an aspect of some embodiments of the present disclosure, there is provided a method of planning implantation of an annuloplasty device to a heart valve, the method including: locating a circumferentially extending portion of a hinge of the heart valve; locating a circumferentially extending portion of a coronary artery; identifying a pathway extending along and between the locations of the two circumferentially extending portions; and providing the identified pathway as a target location for implantation of the annuloplasty device.

According to some embodiments of the present disclosure, the circumferentially extending portion of the hinge is identified automatically, based on measurements of one or more of a geometry of the heart valve, dielectric properties of the heart valve, and electrophysiological signals measured near the heart valve.

According to some embodiments of the present disclosure, the circumferentially extending portion of the hinge is identified by manual selection.

According to some embodiments of the present disclosure, the portion of the coronary artery is identified using electrical field imaging.

According to some embodiments of the present disclosure, the electrical field imaging includes using probes separated by a tissue barrier to map electrical field voltages on either side of the barrier.

According to some embodiments of the present disclosure, the electrical field imaging includes: using a probe moving within a lumen including a valve annulus of the heart valve; and identifying positions at which the probe senses a signal transmitted from within the coronary artery, at an amplitude indicative of close proximity to the coronary artery.

According to some embodiments of the present disclosure, the method includes locating a structure of the heart electrical conduction system; and including adjusting the pathway to remain at least a predetermined distance away from the located structure.

According to an aspect of some embodiments of the present disclosure, there is provided a method of monitoring the implantation of a fastener for an annuloplasty device into a valve annulus, the method including: measuring an electrical signal indicative of impedance using the fastener as an electrode, while the fastener is being brought to an implantation position; providing an indication of fastener position, based on a change in the measured electrical signal.

According to some embodiments of the present disclosure, the fastener is a screw.

According to some embodiments of the present disclosure, the indication indicates fastener contact with tissue of the valve annulus.

According to some embodiments of the present disclosure, the method includes inserting an electrode to a coronary artery; wherein the indication warns of fastener penetration of the coronary artery.

According to some embodiments of the present disclosure, the method includes inserting an electrode to a coronary artery; wherein the impedance is between the fastener and the inserted electrode; and the indication is an indication of fastener proximity to the coronary artery. According to some embodiments of the present disclosure, the indication indicates a depth of fastener penetration into the valve annulus.

According to some embodiments of the present disclosure, the indication of fastener position is also based on the determination that the fastener is located at a valvular position along the axis.

According to an aspect of some embodiments of the present disclosure, there is provided a method of monitoring the implantation of a fastener for an annuloplasty device into a valve annulus, the method including: receiving a specification of implantation positions within a heart posing a risk of damage to a right coronary artery; tracking entry of a fastener to one of the specified implantation positions; and providing, based on the tracked entry, an indication that the fastener is positioned where there is a risk of damage to the right coronary artery.

According to an aspect of some embodiments of the present disclosure, there is provided a method of determining the distance of positions in a first blood-filled lumen from a transmitter probe located in a second blood-filled lumen, the method including: placing a transmitter probe in the second blood-filled lumen; transmitting a signal from the transmitter probe; recording the signal using a sensor positioned on a probe positioned in the first blood-filled lumen; wherein the first and second blood-filled lumens are separated from each other across a barrier of solid tissue; and estimating a distance between the sensor and the transmitter probe, based on an amplitude of the recorded signal.

According to some embodiments of the present disclosure, the transmitted signal includes one or more of an electrical signal, a magnetic signal, and an acoustic signal.

According to some embodiments of the present disclosure, the first blood-filled lumen is a heart chamber, and the second blood-filled lumen is a cardiac artery.

According to some embodiments of the present disclosure, the transmitter probe transmits from a plurality of distinguishable segments along the transmitter probe.

According to some embodiments of the present disclosure, the distance estimation is adjusted according to the segment from which the signal is received.

According to some embodiments of the present disclosure, the distance estimation uses just one of the plurality of distinguishable segments.

According to some embodiments of the present disclosure, the method includes providing a proximity warning, based on the estimated distance.

According to an aspect of some embodiments of the present disclosure, there is provided a system configured to map a vascular lumen extending alongside a body cavity, the system including: a processor, memory storing instructions, and display; wherein the processor is configured to receive respective inputs from: a first probe moving within the body cavity while measuring signals from a plurality of electrical fields, and a second probe through the vascular lumen while measuring signals from the plurality of electrical fields; and wherein the processor operates according to the instructions to: reconstruct a shape of the body cavity and the vascular lumen using the measurements of the first probe and the second probe, and identify vascular lumen positions within the reconstructed shape corresponding to measurement positions of the second probe, and present an image the reconstructed shape on the display with an indication of the vascular lumen position.

According to some embodiments of the present disclosure, the processor further operates to track, based on position measurements received, the position of a potentially traumatizing device as it moves through the body cavity, and estimate the distance of the potentially traumatizing device to the vascular lumen positions.

According to some embodiments of the present disclosure, the potentially traumatizing device is a fastener for an implantable device.

According to some embodiments of the present disclosure, the implantable device is an annuloplasty device.

According to some embodiments of the present disclosure, the processor presents an indication that the potentially traumatizing device is within a potentially hazardous proximity to the vascular lumen.

According to an aspect of some embodiments of the present disclosure, there is provided a system configured to locate a heart valve annulus along an atrioventricular axis, the system including: a processor, memory storing instructions, and display; wherein the processor is configured to receive intracardiac electrophysiological signal waveforms measured from: probe positions extending between an atrial side and a ventricular side of a heart valve annulus, including, for each side, a respective plurality of probe positions; wherein the processor operates according to the instructions to: determine relative spatial locations of the probe positions within the heart, identify, based on the signal waveform measurements, a position and orientation of a region between the atrial side and the ventricular side, the region being positioned at the heart valve annulus along the atrioventricular axis, and oriented to include opposite circumferential sides of the heart valve annulus, and produce a model of the heart.

According to some embodiments of the present disclosure, the spatial locations correspond to locations within a 3-D model of the heart.

According to some embodiments of the present disclosure, the processor further operates to present on the display the 3-D model of the heart marked with the identified spatial locations at the position of the heart valve annulus along the atrioventricular axis.

According to some embodiments of the present disclosure, the processor operates to identify at least one of the spatial locations as being at the position of an atrium along the atrioventricular axis, based on the signal waveform measured at the at least one of the spatial locations.

According to some embodiments of the present disclosure, the processor operates to present on the display a 3-D model of the heart marked with the identified at least one of the spatial locations at the position of the atrium along the atrioventricular axis.

According to some embodiments of the present disclosure, the processor operates to identify at least one of the spatial locations as being at the position of a ventricle along the atrioventricular axis, based on the signal waveform measured at the at least one of the spatial locations.

According to some embodiments of the present disclosure, the processor operates to present on the display a 3-D model of the heart marked with the identified at least one of the spatial locations at the position of the ventricle along the atrioventricular axis.

According to some embodiments of the present disclosure, the processor identifies position along the atrioventricular axis by identifying relative amplitudes of an atrially-generated electrophysiological signal, and a ventricularly-generated electrophysiological signal.

According to some embodiments of the present disclosure, the atrially generated signal includes a P wave of an electrocardiogram.

According to some embodiments of the present disclosure, the ventricularly generated signal includes a QRS complex of an electrocardiogram.

According to some embodiments of the present disclosure, the processor identifies position along the atrioventricular axis by interpolating between atrial-side and ventricular side measurements of the electrophysiological signal waveforms to identify one or more intermediate positions at the heart valve annulus.

According to some embodiments of the present disclosure, the processor identifies position along the atrioventricular axis by identifying a planar region intersecting an entire circumference of the valve annulus.

According to some embodiments of the present disclosure, the processor identifies position along the atrioventricular axis by identifying a non-planar region intersecting an entire circumference of the valve annulus.

According to some embodiments of the present disclosure, the non-planar region is saddle-shaped due to a geometric deformity of the valve annulus.

According to an aspect of some embodiments of the present disclosure, there is provided a system configured to automatically locate a hinge of a heart valve annulus, the system including: a processor and memory storing instructions; wherein the processor is configured to access a 3-D model including: the heart valve annulus, and at least a portion of the heart valve leaflets; and wherein the processor operates according to the instructions to: determine elevation angles of surface orientation relative to the valve annulus along a plurality of radii of the valve annulus; and identify, along the plurality of radii, respective positions that are portions of the hinge, based on elevation angle and/or changes in elevation angle along its respective radius.

According to some embodiments of the present disclosure, the location of the heart valve annulus surface used in determining the elevation angles is at least partially determined based on electrophysiological measurements measured from positions along an atrioventricular axis extending through the heart valve annulus.

According to an aspect of some embodiments of the present disclosure, there is provided a system configured to identifying extents of fibrous tissue within a heart valve annulus, the system including: a processor, memory storing instructions, and display; wherein the processor is configured to receive: measurements of an electrical signal indicating impedance changes, the measurements being obtained from probe positions in proximity to tissue extending from connective tissue of a valve annulus to myocardial tissue surrounding the valve annulus, and relative spatial locations of the probe positions within the heart; and wherein the processor operates according to the instructions to: identify a plurality of the spatial locations as including connective tissue of the valve annulus, based on the measured electrical signal, and present an indication of the identification on the display.

According to some embodiments of the present disclosure, the system includes measuring a time course of changes in the measured electrical signal due to motion of tissue, and identifying a plurality of the spatial locations as including connective tissue of the valve annulus, distinct from leaflets of the valve, based on the measured time course of changes in the electrical signal.

According to an aspect of some embodiments of the present disclosure, there is provided a system configured to detect valve leaflets, the system including: a processor and memory storing instructions; wherein the processor is configured to receive measurements of electrical signals indicative of impedance changes from one or more electrodes located near a cardiac valve; and wherein the processor operates according to the instructions to: determine if the electrode is near a leaflet of the cardiac valve, based on a characteristic of electrical signal changes during a single heartbeat cycle.

According to an aspect of some embodiments of the present disclosure, there is provided a system configured to identify hinge boundary between a heart valve annulus and heart valve leaflets, the system including: a processor, memory storing instructions, and display; wherein the processor is configured to receive respective inputs measuring, at a plurality of probe positions in proximity to one or both of the heart valve annulus and the heart valve leaflets, a time course of changes in an electrical signal indicative of impedance changes due to motion of tissue; and wherein the processor operates according to the instructions to: determine relative spatial locations of the probe positions within the heart, determine motions detected at the plurality of probe positions as characteristic of motions of the valve annulus or of the valve leaflets, determine, as being valve hinge positions, spatial locations between valve annulus locations and valve leaflet locations, and present, using the display, an indication of the identifications of valve hinge positions.

According to some embodiments of the present disclosure, the processor identifies motion-induced electrical signal changes characteristic of the valve leaflets based on a doubled cycle of increasing and decreasing impedance during a single heartbeat cycle.

According to some embodiments of the present disclosure, the location of the heart valve annulus along the atrioventricular axis is determined based on electrophysiological measurements measured from positions along the atrioventricular axis; and the probe positions selected according to the determined location of the heart valve annulus.

According to an aspect of some embodiments of the present disclosure, there is provided a system configured to locate a structure of the electrical conduction system of the heart, the system including: a processor and memory storing instructions; wherein the processor is configured to receive respective measurements of intracardiac electrophysiological signal waveforms made at intracardial probe positions; and wherein the processor operates according to the instructions to: determine spatial locations of the intracardial probe positions within the heart; and identify at least one of the spatial locations as being at the position of the structure, based on the signal waveform measured at the at least one of the spatial locations.

According to some embodiments of the present disclosure, the structure includes a bundle of His, and the signal waveform is characteristic of a latency to waveform arrival at the bundle of His.

According to some embodiments of the present disclosure, the structure includes an AV node, and the signal waveform is characteristic of a latency to waveform arrival at the AV node.

According to an aspect of some embodiments of the present disclosure, there is provided a system configured to plan implantation of an annuloplasty device to a heart valve, the system including: a processor and memory storing instructions; wherein the processor is configured to receive inputs defining: a circumferentially extending portion of a hinge of the heart valve; a circumferentially extending portion of a coronary artery; and wherein the processor operates according to the instructions to: define a pathway extending along and between the locations of the two circumferentially extending portions; and provide the identified pathway as a target location for implantation of the annuloplasty device.

According to some embodiments of the present disclosure, the circumferentially extending portion of the hinge is identified automatically, based on measurements of one or more of a geometry of the heart valve, dielectric properties of the heart valve, and electrophysiological signals measured near the heart valve.

According to some embodiments of the present disclosure, the circumferentially extending portion of the hinge is identified by manual selection.

According to some embodiments of the present disclosure, the portion of the coronary artery is identified using electrical field imaging.

According to some embodiments of the present disclosure, the electrical field imaging includes the use of measurement from probes separated by a tissue barrier to map electrical field voltages on either side of the barrier.

According to some embodiments of the present disclosure, the electrical field imaging includes: use of a probe moving within a lumen including a valve annulus of the heart valve; and identification of positions at which the probe senses a signal transmitted from within the coronary artery, at an amplitude indicative of close proximity to the coronary artery.

According to some embodiments of the present disclosure, the processor is instructed to receive a location of a structure of the heart electrical conduction system; and adjust the pathway to remain at least a predetermined distance away from the located structure.

According to an aspect of some embodiments of the present disclosure, there is provided a system configured to monitor the implantation of a fastener for an annuloplasty device into a valve annulus, the system including: a processor, memory storing instructions, and display; wherein the processor is configured to receive measurements of an electrical signal indicative of impedance using the fastener as an electrode, while the fastener is being brought to an implantation position; and wherein the processor operates according to the instructions to present to the display an indication of fastener position, based on a change in the measured electrical signal.

According to some embodiments of the present disclosure, the fastener is a screw.

According to some embodiments of the present disclosure, the indication indicates fastener contact with tissue of the valve annulus.

According to some embodiments of the present disclosure, the indication warns of fastener penetration of the coronary artery.

According to some embodiments of the present disclosure, measurements are of impedance between the fastener and an electrode inserted to the coronary artery; and the indication is an indication of fastener proximity to the coronary artery.

According to some embodiments of the present disclosure, the indication indicates a depth of fastener penetration into the valve annulus.

According to some embodiments of the present disclosure, the processor receives an estimated position of the fastener along an axis between an atrium and a ventricle; and the indication of fastener position is also based on the determination that the fastener is located at a valvular position along the axis.

According to an aspect of some embodiments of the present disclosure, there is provided a system configured to monitor the implantation of a fastener for an annuloplasty device into a valve annulus, the system including: a processor and memory storing instructions; wherein the processor is configured to receive: a specification of implantation positions within a heart posing a risk of damage to a right coronary artery, and position data indication positions of a fastener moving within the heart; wherein the processor operates according to the instructions to: track entry of the fastener to one of the specified implantation positions; and provide, based on the tracked entry, an indication that the fastener is positioned where there is a risk of damage to the right coronary artery.

According to an aspect of some embodiments of the present disclosure, there is provided a system configured to determine the distance of positions in a first blood-filled lumen from a transmitter probe located in a second blood-filled lumen, the system including: a processor, memory storing instructions, and display; wherein the processor is configured to receive a signal: sensed by a sensor positioned on a probe positioned in the first blood-filled lumen, and transmitted to the sensor from a transmitter probe in the second blood-filled lumen; wherein the processor operates according to the instructions to estimate a distance between the sensor and the transmitter probe, based on an amplitude of the received signal; and wherein the first and second blood-filled lumens are separated from each other across a barrier of solid tissue.

According to some embodiments of the present disclosure, the transmitted signal includes one or more of an electrical signal, a magnetic signal, and an acoustic signal.

According to some embodiments of the present disclosure, the first blood-filled lumen is a heart chamber, and the second blood-filled lumen is a cardiac artery.

According to some embodiments of the present disclosure, the transmitter probe transmits from a plurality of distinguishable segments along the transmitter probe.

According to some embodiments of the present disclosure, the distance estimation is adjusted according to the segment from which the signal is received.

According to some embodiments of the present disclosure, the distance estimation uses just one of the plurality of distinguishable segments.

According to some embodiments of the present disclosure, the processor presents, using the display, a proximity warning, based on the estimated distance.

According to an aspect of some embodiments of the present disclosure, there is provided a method of monitoring a structural heart disease intervention including introduction of a device into a heart chamber, the method including: accessing a structural representation of a portion of a heart; accessing electrophysiological measurements indicating electrical activity of tissue of the heart; associating the electrophysiological measurements to locations in the structural representation of the portion of the heart; presenting an image of the structural representation of the portion of the heart, together with indications of values of the electrophysiological measurements at their associated locations.

According to some embodiments of the present disclosure, the structural representation is a three-dimensional structural representation.

According to some embodiments of the present disclosure, the electrophysiological measurements comprise electrophysiological measurements recorded using the device.

According to some embodiments of the present disclosure, the method includes estimating a position of the device, using the electrophysiological measurements recorded using the device.

According to some embodiments of the present disclosure, the presented indications of the values of the electrophysiological measurements comprise identifications of different tissue structures associated with the electrophysiological measurement.

According to some embodiments of the present disclosure, the method comprises presenting an indication of a location to be avoided for attachment of the device, wherein the avoided location is determined based on the electrophysiological measurements.

According to some embodiments of the present disclosure, the avoided location is determined based on electrophysiological measurements at the location corresponding to electrophysiological characteristics of a bundle of His.

According to some embodiments of the present disclosure, the avoided location is determined based on electrophysiological measurements at the location corresponding to electrophysiological characteristics of an AV node.

According to some embodiments of the present disclosure, the device is an implantable annuloplasty device.

According to an aspect of some embodiments of the present disclosure, there is provided a method of guiding a structural heart disease intervention, including: accessing a structural representation of a heart; accessing electrophysiological measurements indicating electrical activity of tissue of the heart; associating the electrophysiological measurements to locations in the structural representation of the heart corresponding to locations at which the measurements were recorded; selecting a location for attachment of a device configured to provide structural heart disease intervention, based on the structural representation, the electrophysiological measurements, and their locations in the structural representation; and presenting an image of the structural representation of the heart wherein the selected location is marked.

According to an aspect of some embodiments of the present disclosure, there is provided a method of verifying a structural heart disease intervention, including: accessing a structural representation of a heart; accessing electrophysiological measurements obtained from locations within the heart before and after implantation to the heart of a device configured to provide a structural heart disease intervention; associating the electrophysiological measurements to locations in the structural representation of the heart corresponding to locations at which the measurements were recorded; and comparing electrophysiological activity before and after implantation of the device in at least one location of the heart to check for impairment of electrophysiological activity at the at least one location.

According to an aspect of some embodiments of the present disclosure, there is provided a method of guiding a structural heart disease intervention, including: accessing electrophysiological (EP) measurements of the heart measured from a specified location; and guiding the structural heart disease intervention based on the accessed EP measurements.

According to some embodiments of the present disclosure, guiding the structural heart diseases intervention includes indicating on an image of a portion of the heart a current location of an implant for use in the intervention, the specified location, and the accessed EP measurements. Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a schematic flowchart of a method of guiding and monitoring implantation of a tricuspid heart valve annuloplasty device using a multimodal measurement approach, according to some embodiments of the present disclosure;

FIGS. 2A-2G schematically illustrate selected phases in the implantation of annuloplasty device, together with examples of auxiliary tools used to guide and/or monitor the implantation, according to some embodiments of the present disclosure.

FIG. 3A schematically illustrates implantation of an annuloplasty device for treatment of regurgitation in a mitral valve, according to some embodiments of the present disclosure.

FIG. 3B is a schematic flowchart of a method of guiding and monitoring implantation of a mitral heart valve annuloplasty device, according to some embodiments of the present disclosure;

FIG. 4A schematically represents an overhead view (looking from a right atrium toward a right ventricle) of a tricuspid valve, according to some embodiments of the present disclosure;

FIGS. 4B-4C schematically illustrate examples of displays used in device implantation planning and/or in performing device implantation, according to some embodiments of the present disclosure;

FIG. 5A schematically represents a graph of impedance changes as a function of time (in seconds) as measured between electrode pairs of a multi-electrode probe (e.g., a lasso-type catheter) positioned in the right atrium and generally “above” the tricuspid valve leaflets along an atrioventricular axis, according to some embodiments of the present disclosure;

FIG. 5B schematically represents time traces of respiration (trace), and body surface ECG (trace), according to some embodiments of the present disclosure;

FIG. 6 schematically represents an annuloplasty device, according to some embodiments of the present disclosure;

FIG. 7A schematically illustrate a method of identifying valve hinge locations, according to some embodiments of the present disclosure;

FIG. 7B schematically illustrates a method of using time-frequency decomposition to distinguish components of heart structure as belonging to different structures, according to some embodiments of the present disclosure;

FIG. 8 schematically represents detection of wall contacts, according to some embodiments of the present disclosure; and

FIG. 9 is schematic diagram of a system for monitoring and/or guiding annuloplasty device implantation, according to some embodiments of the present disclosure.

FIG. 10A schematically illustrates coronary artery proximity and penetration by a device fastener, according to some embodiments of the present disclosure; and

FIG. 10B schematically represents features of coupling measurements potentially useful to detect changes of coronary artery proximity and penetration by a fastener, according to some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

An aspect of some embodiments of the present disclosure relates to the integration of multimodal measurements of an intrabody environment of a medical procedure—including measurements of structure and of events taking place therein—into a compound model which unifies them. The compound model may be used, in some embodiments, to guide and/or monitor the procedure. More particularly, the present disclosure describes guidance and monitoring of heart valve annuloplasty procedures; for example, performed upon the tricuspid valve and/or the mitral valve of a human heart. It should be understood that while overall descriptions are provided in terms of heart valve annuloplasty procedures, there are also described general principles which relate to structural heart disease intervention more generally.

The term “multimodal measurement” refers to the use of measurements of a plurality of different types to characterize the intrabody environment of the procedure and/or activities taking place within it. The prefix “multi-” should be understood to apply to the overall approach, which is capable of integrating measurements made using several different approaches into a compound model of the intrabody environment. Any individual embodiment of the present disclosure optionally uses a particular set of a plurality of measurement approaches.

Embodiments of the present disclosure may be understood as establishing a “scaffolding” that is built (e.g., based on earlier measurements in a procedure, or inputs from pre-procedure data sources) to provide a basic model of a procedure's environment. Further measurements continuously provide further detail to and/or update that model, as they are associated to their appropriate places within the compound model. The compound model develops over time as a result. The association of new measurements is performed in a manner that supports guiding and/or monitoring a procedure in real time.

The scaffolding of the basic model is optionally based on a primary measurement modality (for example, imaging by electrical field measurements and/or ultrasound intra-cardiac echocardiography, ICE, MRI, and/or CT), or built up by the coordinated use of a plurality of measurement methods (and thus may be “compound” from the beginning). Measurements may be made using devices auxiliary to the annuloplasty device (e.g., imaging probes placed within the heart lumen or at other locations). Optionally, the annuloplasty device itself is used as a measurement device; for example, conductive elements of the device such as control wires and/or fasteners are configured as electrodes, and/or electrodes are attached to the device and/or positioned where they have a known spatial relationship to the device (e.g., on a sheath of a delivery catheter of the device).

Locations within the compound model are optionally described in terms of spatial coordinates (spatial positions) and/or distances; and/or in terms of non-spatial metrics which characterize a measurement, such as its signal phase, amplitude, and/or eigenvalue of one or more eigenvector components of the measurement (e.g., as determined by a method of mathematical decomposition). In some embodiments, a compound model includes both spatial and non-spatial representations. For example, electrophysiological measurements may be used to guide a procedure by the assessment of “similarity (of the measurement) to a target”, while “spatial distance to a target” may be provided in coordination with the similarity assessment, e.g., to confirm and/or refine it.

Relating to annuloplasty procedures in particular: embodiments of the present disclosure describe multimodal measurement-based solutions for problems which arise during the course of valvular annuloplasty, including problems associated with:
locating and/or identifying a region targeted for implantation of an annuloplasty device;
planning and/or actual implantation of the device which avoids damage to sensitive heart areas; and/or
verification of attachment of the device to the heart.

A particular class of multimodal measurement-based compound models combines electrophysiological measurements of heart activity with detailed positional (including detailed structural shape) information. In some embodiments, intracardiac measurements of endogenous electrical activity are localized in space by coordinating them closely with locations (e.g., the “scaffolding”) defined by the compound model. Optionally, the locations are themselves further characterized according to their suitability as sites for annuloplasty device attachment, e.g., based on a determination that they comprise fibrous tissue of the valve annulus. The electrophysiology reveals, in some embodiments, locations the implantation should avoid (e.g., because certain electrically active tissue in the locations to be avoided is particularly vulnerable to mechanical damage). This information is optionally used to exclude device attachment at otherwise (e.g., mechanically) suitable attachment sites.

Compound models are optionally displayed as images, for example images which combine structural anatomy with functional anatomy such as electrophysiological measurement results, and optionally computer-processed interpretations of electrophysiological measurements: for example, the location of the AV node, the location of the bundle of His, the location of a heart valve structure such as the valve annulus, and/or position along an axis over which the electrophysiological measurements themselves vary, for example as a function of waveform component amplitude and/or timing.

Potential advantages of applying a multimodal measurement approach to valve annuloplasty is a reduction in how aggressive, risky, and/or expensive the overall procedure is. For example, in some embodiments, multimodal measurement provides information sufficient to guide the procedure without the use of methods that are performed with general anesthesia; for example, trans-esophageal ultrasound imaging. In turn, when general anesthesia is avoided, a requirement for artificial ventilation is potentially removed. Apart from adding to the complexity of the procedure, artificial ventilation has the effect of changing normal negative pressure breathing (sucking air in via movements of the chest and diaphragm) into positive pressure breathing (pushing air in artificially). Positive pressure, in turn, can have an effect on the shape of the heart, including shrinking valves that are normally more open and prone to regurgitation. Consequentially, an annuloplasty procedure performed during positive pressure ventilation potentially under-corrects; or if positive pressure effects are taken into account, may paradoxically over-correct.

In some embodiments of the present disclosure, multimodal measurement removes a need to obtain a prior spatial map of the heart using CT or MRI imaging. Potentially, a need for planned open-heart surgery procedures and/or a risk of complications which lead to unplanned open-heart surgery procedures is reduced.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Annuloplasty Device Implantation

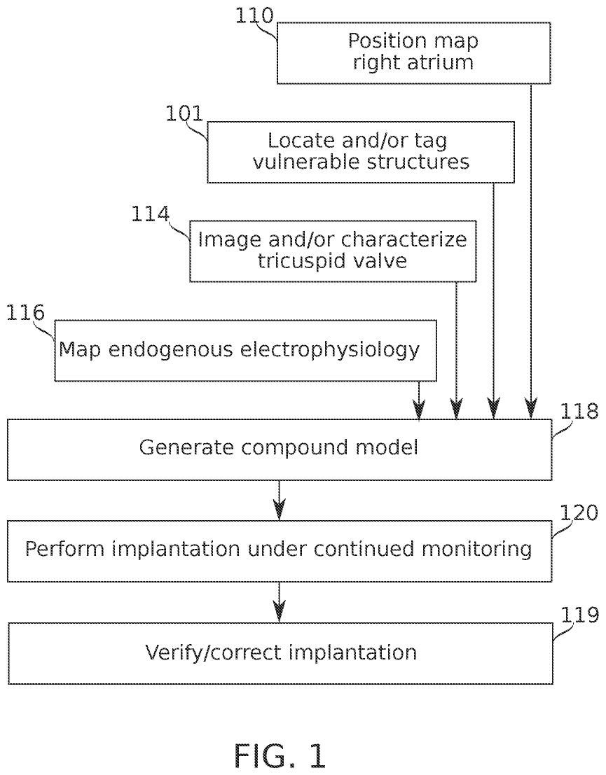

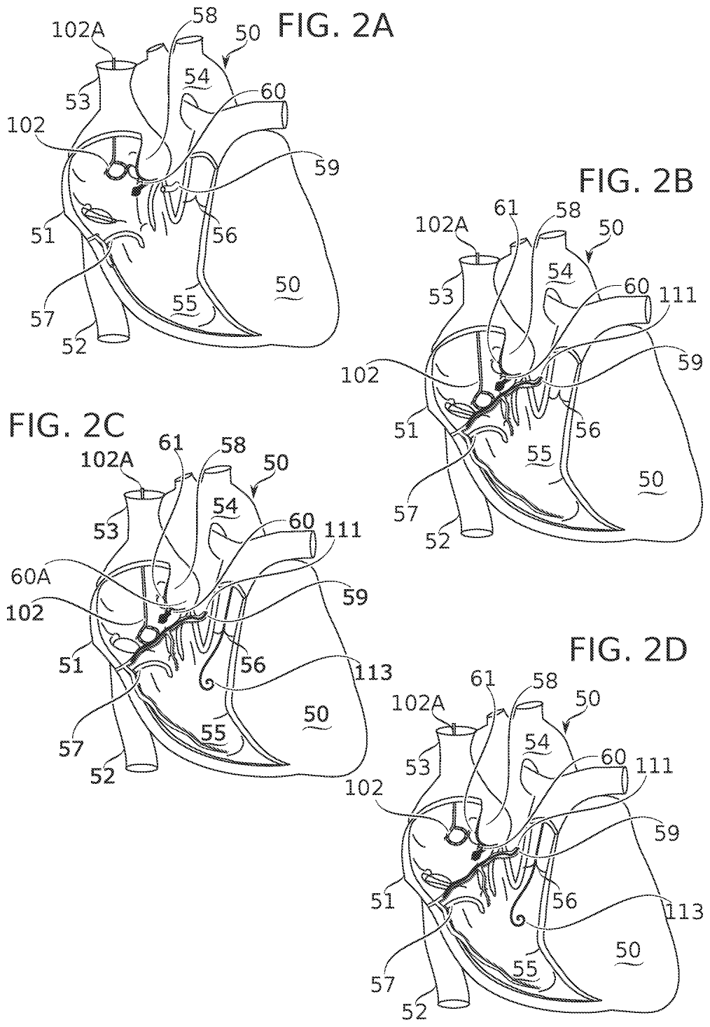

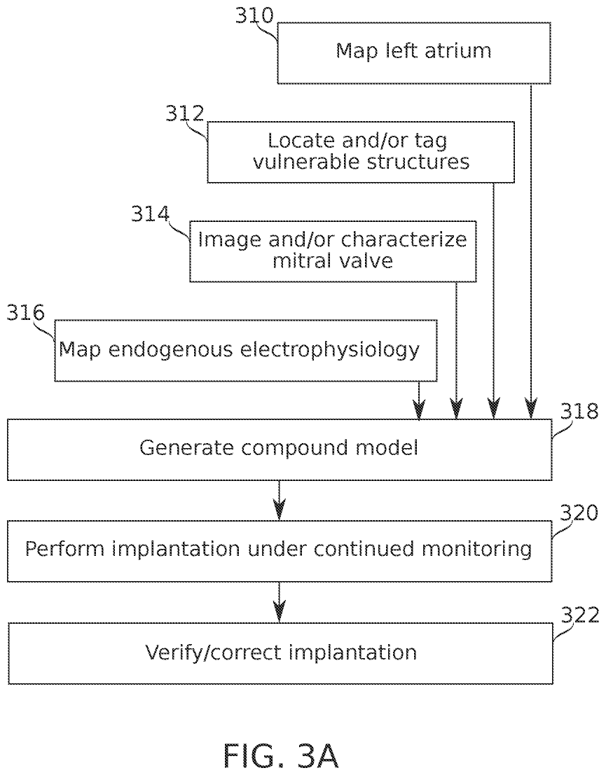

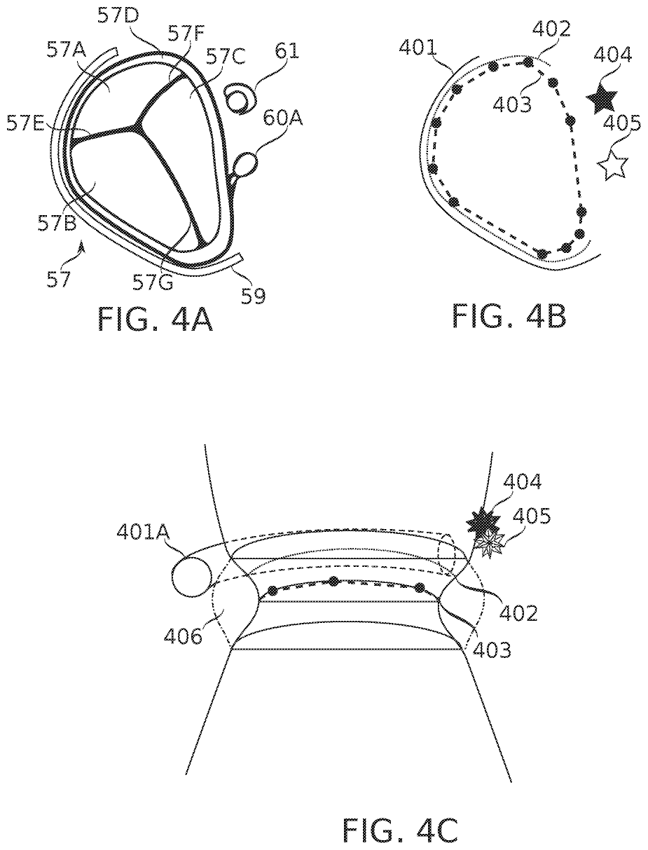

Reference is now made to FIG. 1, which is a schematic flowchart of a method of guiding and monitoring implantation of a tricuspid heart valve annuloplasty device 112 using a multimodal measurement approach, according to some embodiments of the present disclosure. Reference is also made to FIG. 6, which schematically represents an annuloplasty device 112, according to some embodiments of the present disclosure. Additional reference is made to FIG. 4A, which schematically represents an overhead view (looking from a right atrium 51 toward a right ventricle 55) of a tricuspid valve 57, according to some embodiments of the present disclosure. Further reference is made to FIGS. 2A-2G, which schematically illustrate selected phases in the implantation of annuloplasty device 112, together with examples of auxiliary tools used to guide and/or monitor the implantation, according to some embodiments of the present disclosure.

An implantable device used in tricuspid annuloplasty, in some embodiments, comprises annuloplasty device 112 (e.g., as shown in FIG. 6), which is attached to tissue extending around a circumference of a tricuspid valve 57, and then actuated (e.g., altered in shape by shrinking) to modify function of the tricuspid valve 57. The modification is aimed at reducing regurgitation through the valve. In some embodiments, the attachment is performed by inserting fasteners 122 (e.g., screws, coils, or another anchoring device) into tissue surrounding the tricuspid valve 57, for example from within a sleeve 121 of the device. Actuation of the annuloplasty device 112, in some embodiments, comprises cinching of cord 125. This causes the circumference of annuloplasty device 112 to reduce. In accord with this, the circumference of tricuspid valve 57 itself is reduced (optionally after a period of remodeling). This potentially reduces regurgitation through tricuspid valve 57 by bringing the leaflets 57A-57C into closer apposition (coaptation).

The implantation is performed, in some embodiments, using minimally invasive (e.g., over-catheter) techniques of delivery, positioning, deployment, and/or attachment. Approaches to the heart for minimally invasive procedures include, for example: vascular approaches via the inferior or superior vena cava; through arteries (e.g., from the carotid artery or by small chest incision); or in some embodiments through the apex of the left ventricle.

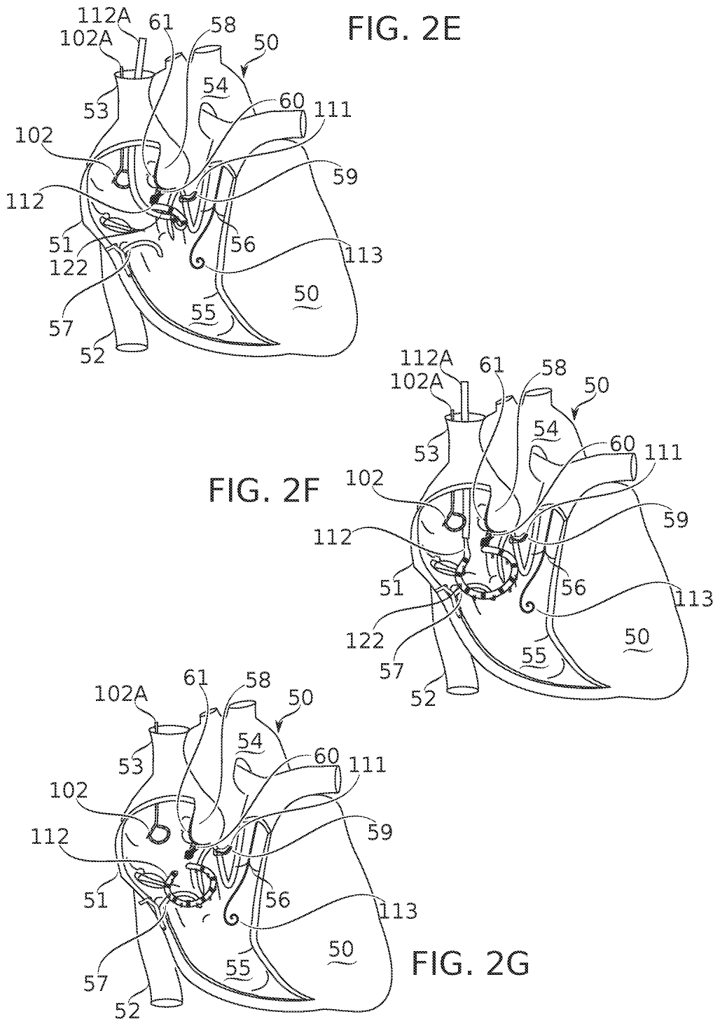

A range of problems (described further in the descriptions following) are associated with implantation of annuloplasty devices (e.g., the tricuspid valve annuloplasty method of FIG. 1, and/or the mitral valve method annuloplasty method of FIG. 3A). These problems potentially interfere with safety, reliability, and/or effectiveness of the device and/or the procedure which implants it. In some embodiments, problems are potentially mitigated by the measurement and use of data which indicate aspects of the anatomical and functional environment of the device at the site of implantation, and/or aspects of the device itself.

With respect to the anatomical/functional environment, data may indicate, for example, overall heart lumen shape, overall heart function, heart structural anatomy, and/or heart functional anatomy. Although these categories are not dichotomous (the same data potentially belongs to more than one of these categories), the categories mentioned represent differences in emphasis.

In particular, data indicating heart structural anatomy optionally encompass one or both of (for example):

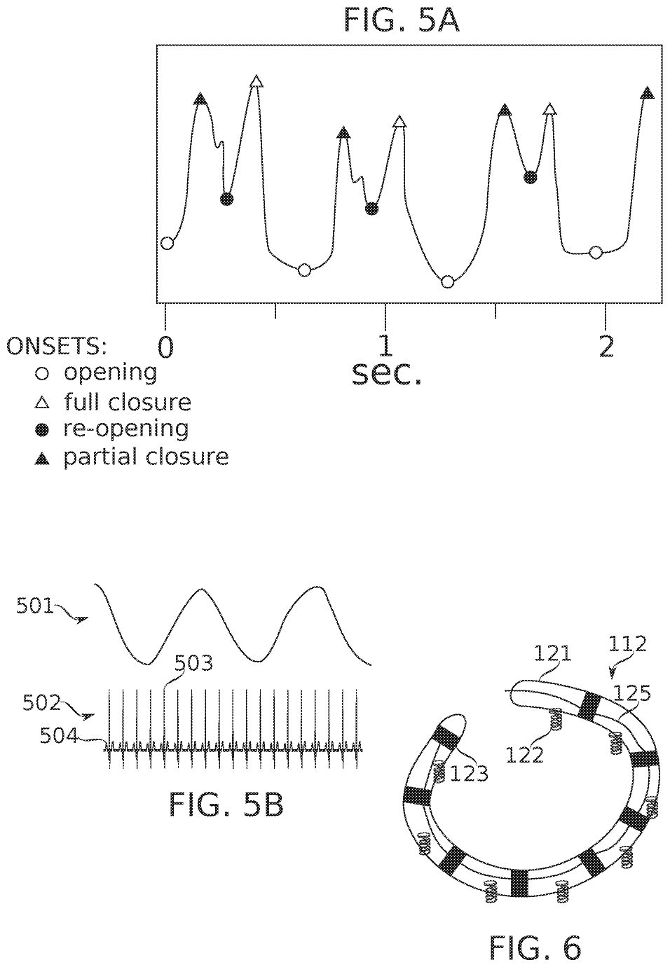

Locations of regions and/or boundaries of heart tissue, defined based on distinctions in mechanical and/or cellular-level properties. These optionally include, for example: the boundary between fibrous tissue of the valve annulus 57D and surrounding cardiac muscle, the boundary between valve leaflets and the valve annulus 57D, and/or the course of transmission pathways such as the bundle of His 60. FIG. 7A provides an example.

Specifics of cardiac shape including the detailed shape and/or location of lumenal and/or perilumenal structures. These optionally include, for example: papillary muscles, chordae, valve leaflets, valve annuli, cardiac structures specialized for impulse transmission (e.g., sinoatrial (SA) node, atrioventricular (AV) node 60A (for example, as described in relation to FIGS. 2C and/or 4A), and/or bundle of His 60), blood vessels supplying and draining the cardiac tissue (coronary arteries, coronary veins), and/or other major blood vessels.

While some of these may be deduced in part from overall heart lumen shape, “overall heart lumen shape” as such refers herein to the (optionally time-varying) shape of the lumenal wall boundary as such (e.g., the bounds of movement of an object within the lumen), without specific reference to tissue properties.

Data indicating heart functional anatomy optionally encompass one or more of (for example):

Passive and/or active details of how structures move; for example, movements of valve leaflets and/or contractions of cardiac muscle.

Electrical activity of cardiac tissue, optionally including variations over time and space.

Measurements that provide a metric for a function attributable to a specific heart structure:

for example, a measurement of the backward flow of blood through the defective tricuspid valve (known as tricuspid valve regurgitation) may characterize the functional anatomy of the tricuspid valve 57, not necessarily with structural detail. The tricuspid valve regurgitation may be measured, for example, by sensing backflow of an injected tracer fluid such as saline or dye.

Metrics of anatomy and/or movement associated with specific structures, and carrying special meaning for the operation of the heart. For example, an open area of the tricuspid valve 57 when it is maximally closed is structural, but also a metric of regurgitation. In another example: a percentage of shortening of the papillary muscles during the cardiac cycle (summarizing their dynamic motion) has potential implications for valve function such as risk of prolapse.

In contrast to such structurally-associated data, data characterizing “overall heart function” includes, for example: body surface ECG recordings, heart rate, and/or overall pumping volume of the heart.

Herein, a measurement modality processes measurement data with some processing device and/or method, thereby producing processed measurements particular to the measurement modality. Herein, the data may include, for example, indications of structural anatomy, functional anatomy and/or overall heart function. One or more analysis procedures are applied to extract specific information from the data; for example, measurements of electrophysiological function may be processed to identify positions of certain heart structures. Measurement modalities are not necessarily segregated from each other in both data and processing. For example, in some embodiments, the same tool (e.g., electrode-carrying probe), and optionally even the same stream of raw measurements (e.g., a stream of voltage measurements from an electrode of the electrode probe) is used as the basis of a plurality of measurement modalities. In such embodiments, the measurement modalities are distinguished from each other, for example, by different algorithmic processing, by auxiliary information used, and/or by how outputs are integrated to the multimodal model and/or presented for display. It should be understood, however, that measurement modalities are optionally grouped together according to their commonalities; for example, the measurement device and/or source of signal energy being measured (for example: electrodes/electrical fields, magnets/magnetic fields, ultrasound transducer/ultrasound generator, X-ray sensor/X-ray source). Blocks 110, 101, 114, and 116 of FIG. 1 (and corresponding blocks 310, 312, 314, and 316 of FIG. 3A) relate to certain specific measurement modalities. Integration of measurement modalities (to the compound model) is further discussed herein, particularly with reference to block 118 of FIG. 1 and/or block 318 of FIG. 3A.

With respect to the device itself: data may indicate, for example: device position, device orientation, device deployment status, and/or device attachment status. Data may also indicate, directly or indirectly, how the device interacts with the anatomical environment, e.g., to affect (actually, as estimated, and/or as predicted) cardiac function. The interactions measured are potentially intended and/or unintended; therapeutic and/or adverse. Herein, measurements related to device status are discussed with reference, for example, to blocks 120, 119 of FIG. 1, and/or blocks 320, 322 of FIG. 3A.

In some embodiments of the present disclosure, implantation and/or validation of implantation is guided and/or monitored using within-body devices for imaging and/or sensing. In some embodiments, this is without the use of ionizing radiation (e.g., without the use of X-ray and/or radionuclide-based imaging).

A particular potential advantage provided by some embodiments of the present disclosure is conferred by use of electrode measurements to provide a plurality, and optionally substantially all, of the data used in the different measurement modalities. For example, an electrode used in mapping positions within a cardiac lumen is optionally used also for sensing:
differences in dielectric properties characteristic of different tissue structures, and/or contact therewith,
dielectric signals characteristic of an injected tracer (such as saline),
intrinsic cardiac electrical activity, and/or
electrical signals transmitted by a marker device such as a wire inserted to the coronary artery 59.

Potential advantages of using electrical measurements, compared to other intrabody probe types, include reduced numbers of probes, and/or increased simplicity of probes. Optionally, the implantation catheter 112A (FIGS. 2E-2F; FIG. 3B) and/or the implanted annuloplasty device itself and/or its attachment hardware includes at least some of the measurement electrodes, for example, electrodes positioned along a body of the catheter, potentially reducing a number of catheters to be inserted during the procedure. There is also a potential advantage for data integration, for example, insofar as data for two different measurement modalities can be directly identified as characterizing the same location, for example, if they were recorded by the same probe at the same time. There are also potential advantages in terms of implementation, by reducing complexity in coordinating between disparate measurement devices.

Some descriptions herein relate to a respective operation (action/s within a procedure) performed in a certain manner, to achieve some particular intermediate result of an overall implantation procedure. It is to be understood that such operations are optionally performed within any suitable overall procedure, and performed, moreover, in any suitable combination with other operations of the procedure, and in any suitable order, as may be selected to achieve the implantation. For example, some operations are described as one option among a plurality of options for accomplishing the same intermediate result; any procedure which includes accomplishing that intermediate result optionally uses any of the plurality of options. In some embodiments, the intermediate result itself is an optional part of the overall procedure—for example, an operation to verify position and/or attachment may be performed optionally. Moreover, in some embodiments of the present disclosure, all or portions of operations optionally occur sequentially (and the particular order of the sequence itself is optionally defined) and/or in parallel (e.g., simultaneously) with each other. This applies, in particular but not only, to operations described in relation to blocks 110, 101, 114, and/or 116 of FIG. 1 (and/or blocks 310, 312, 314 and/or 316 of FIG. 3A).

FIG. 1 describes general classes of operations which are optionally performed in some embodiments of the present disclosure, relating them generally to measuring, mapping, and positioning devices. FIGS. 2A-2D (and their associated descriptions) relate more specifically to operations which measure and help define the anatomical environment (and may comprise instances of operations of FIG. 1). FIGS. 2E-2G (and their associated descriptions) relate more specifically to operations during the implantation of an annuloplasty device 112, and/or to validation of the implantation (which may comprise instances of operations of FIG. 1).

### Position Mapping

At block 110, in some embodiments, the right atrium 51 is measured, and the measurements converted to a position map indicating the shape of at least portions of the internal lumen of right atrium 51. Optionally, position measurements of the tricuspid valve 57 and/or right ventricle 55 are also made within the actions of block 110 (although measurements of tricuspid valve 57 are related to more specifically in relation to block 114).

The position map, in some embodiments, defines (e.g., is used to estimate by computer-implemented processing) a shape of an interior surface of one or more cardiac lumens of a heart. In some embodiments, the position map defines the shape of a volume limited by interior surfaces of the one or more cardiac lumens. The position map is optionally dynamic (e.g., defined as a function of heartbeat and/or respiratory phase) and/or processed (e.g., from dynamic data) to produce a static position map (for example, by use of gating and/or a process of frequency component decomposition; e.g., as described in relation to FIG. 7B, herein).

Position mapping comprises, in some embodiments, movement of a multi-electrode catheter probe 102 (FIG. 2A) within the lumen of, for example, the right atrium 51, while making electrical measurements (e.g., of voltage, current, and/or impedance) of a plurality of exogenously generated (that is, artificially generated) electrical fields which “tag” the volume within which the electrode catheter probe moves, and/or establish an electric field-defined coordinate system within the heart. The electrical fields are optionally generated at different frequencies, and/or multiplexed in time so that they can be distinguished from each other. Each electrical field is generated using an electrode set comprising a plurality of electrodes; the sets of electrodes used for generating the different electrical fields are at different body surface and/or internally situated positions, so that the resulting electrical fields have gradients crossing within the heart in different directions. This causes a different set of measurements to be obtained at each measurement location, such that the set of measurements is characteristic of the location.

The shape of the lumen, in some embodiments, is modeled by a process of reconstructing positions from the measurements. Optionally, a plurality of electrodes of the multi-electrode catheter 102 are operated to make simultaneous measurements. Optionally, reconstructing the positions comprises making use of known inter-electrode distances to constrain a solution which associates each set of measurements with a particular position in space, for example as described in International Patent Publication Nos. WO 2019/035023 A1, entitled FIELD GRADIENT-BASED REMOTE IMAGING; WO 2018/130974 A1, entitled SYSTEMS AND METHODS FOR RECONSTRUCTION OF INTRA-BODY ELECTRICAL READINGS TO ANATOMICAL STRUCTURE; and/or WO 2019/034944 A1, entitled RECONSTRUCTION OF AN ANATOMICAL STRUCTURE FROM INTRABODY MEASUREMENTS—the contents of each of which are included by reference herein, in their entirety.

In some embodiments, another method is used to position map the right atrium 51 and optionally associated structures such as the left ventricle 55. For example, mapping is performed using a different electrical field imaging method, ultrasound, X-ray imaging (fluoroscopy), CT imaging, MRI imaging, and/or magnetic field imaging. X-ray images, for example, may be used to establish a background within which positions and movements of other procedure elements (e.g., catheters, probes, and/or the annuloplasty device 112 itself) are visualized during the procedure. In the case of ultrasound imaging, a probe at the illustrated position of multi-electrode probe 102 may optionally or additionally comprise an ultrasound imaging probe.

In the case of magnetic imaging, a probe at the illustrated position of multi-electrode probe 102 may optionally or additionally comprise a magnetic imaging sensor (e.g., coil). In the case of electrical field imaging methods, a probe at the illustrated position of multi-electrode probe 102 may optionally be a loop (lasso) probe such as is illustrated, or another multi-, dual- or single-electrode catheter probe, for example one with electrodes arranged along a corkscrew (which potentially provides an increased sensitivity to depth along an axis parallel to the longitudinal axis of the corkscrew), or a catheter probe configured closer to a wheel-and-axle configuration, with the loop of the “wheel” approximately centered on the main body of the probe.

In some embodiments, the position-measuring probe is positioned outside the heart (e.g., inside the esophagus in the case of transesophageal echocardiography (TEE)), and optionally outside the body (e.g., on the chest in the case of transthoracic echocardiography (TTE)). It is noted that successful imaging with ultrasound imaging techniques is difficult to achieve in certain cases, for example due to a patient-specific configuration of anatomy that interferes with visualization. In some embodiments, the position measuring probe is positioned in the coronary artery.

The position mapping of block 110, in some embodiments, is expanded to include all or portions of other heart chambers, blood vessel portions, and/or other heart structures for example, right ventricle 55, inferior vena cava 52, superior vena cava 53, and/or tricuspid valve 57. Optionally, left ventricle 42, and/or left atrium 49 are position mapped additionally/instead; for example for performing a mitral valve annuloplasty, for example as described in relation to FIGS. 3A-3B.

The position map of block 110, in some embodiments, provides a kind of “scaffold”, to the positions of which other measurement data (for example, measurement data as described in relation to blocks 101, 114, and/or 116) may be associated (the operation of association is described further, for example, in relation to block 118).

### Localization of Vulnerable Structures

At block 101, in some embodiments, anatomical structures of special concern for damage during a procedure are located and/or tagged. These may also be considered areas to avoid during implantation. A primary target in annuloplasty is the valve itself, and more particularly, for purposes of attachment, the fibrous tissue area of the valve annulus. Characterization of the valve is detailed further in relation to block 114. However, this target is potentially in proximity to one or more areas with particular vulnerabilities, and it is a potential advantage to know clearly where an annuloplasty device is relative to these structures.

As examples, these areas with particular vulnerabilities include, in some embodiments, the AV node 60A, the bundle of His 60, and the right branch of the coronary artery 59. Damage to the AV node 60A and/or bundle of His 60 can result in complete or partial block (e.g., AV block), which can occur in different degrees of severity, up to and including cardiac arrest and possible death. Both of these structures are near enough to the tricuspid valve that a significant risk of complication exists: an annuloplasty implant fastener could cause acute damage, and/or pressure from the annuloplasty device 112 itself (and/or fastener) might eventually induce damage and resulting block of cardiac impulse transmission. Puncture of the coronary artery 59 is another severe complication; bleeding and loss of heart perfusion may require unplanned interventions to manage, and death is again a potential outcome.

Accordingly, it is a potential advantage to locate these structures by whatever means are available, and then, in some embodiments, continue to track their location relative to ongoing procedure activities, to help ensure that damage does not occur, and/or to guide earlier activities so that the procedure does not later on reach an impasse, or require extraordinary measures to avoid causing damage to sensitive heart areas. In some embodiments, structures of the electrical conduction system of the heart (such as the bundle of His or the AV node) are identified by the relative time of waveform arrival to their location, compared, e.g., to surrounding tissue, and/or a reference location such as the sinoatrial node.

In some embodiments, the position of the coronary artery 59 is “tagged” by inserting a catheter wire 111 into it, e.g., via access from the coronary artery 59. In FIG. 2B coronary artery 59 wraps around an exterior portion of the anterior wall of the right atrium and ventricle which is shown cut away in the drawings) Catheter wire 111 may comprise, for example, an electrical wire, or another thin, longitudinally extended probe, such as an electrode microcatheter. The catheter wire 111 can be driven with any suitable pattern of electrical and/or magnetic activation. Additionally or alternatively, wire 111 is acoustically driven (i.e., vibrated). The resulting signal transmits across the tissue barrier(s) separating the lumen of the coronary artery from the lumen of the right atrium and/or ventricle. Locations where the activation pattern is sensed strongly (e.g., by an electrode probe 102, and/or a suitable acoustic transducer) are then recorded as being close to the coronary artery 59 (optionally with a distance decreasing with correspondingly increasing sensed amplitude). It should be understood that the method is optionally carried out with a catheter probe comprising a plurality of separate electrodes (or other transmitters, for example, acoustic or magnetic transmitters) in lieu of a single wire. The transmitters are optionally driven all together (by the same potential), or separately; e.g., with separately distinguishable frequencies and/or periods of transmission.

Locations within the right atrium 51 and/or right ventricle 55 where the signal is strongest (largest amplitude) represent locations nearer to the coronary artery 59 which are preferably avoided by potentially traumatic implantation activities like fastener attachment. The example of a fastener is used herein, but it should be understood that any object (herein, a “potentially traumatizing device”) which has the potential to transmit harm by penetration, pressure, heating, or other trauma, and can be tracked, is optionally monitored and/or guided as is described for the example of a fastener. Examples of such devices include RF ablation probes, needles, cryoablation probes, and implanted devices of any type which exert potentially harmful pressure onto tissue when implanted. In some embodiments, positions within some specification of estimated distance and/or of signal amplitude are considered as being in hazardous proximity to the coronary artery 59; and optionally an indication is provided to the user (e.g., a visual, auditory, and/or haptic alert) that the zone of hazardous proximity has been entered. The zone may be determined by a distance/signal amplitude threshold, and/or determined according to additional considerations such as the type and/or estimated orientation of a tracked device (e.g., the direction of a pointed end of the device) that is approaching the zone of hazardous proximity. In particular, fasteners which operate as anchors by penetration of tissue may be tracked (e.g, configured to be used as electrodes making measurements from which position can be estimated absolutely and/or distance relative to the zone of hazardous proximity can be estimated), and monitored for entry into a zone of hazardous proximity.

It should be understood that distances may be measured between any combination of two adjacent body lumens, e.g., body lumens which are both blood-filled (heart chambers and/or blood vessels). In particular, a transmitting catheter wire or other long transmitting probe placed within a longitudinally extended and radially narrow lumen (e.g. a blood vessel) provides a potential advantage insofar as the transmitter can be operated as a single unit to “tag” a long longitudinal extent of the lumen.

In embodiments wherein transmission is distinguishable among a plurality of transmitters distributed along a catheter probe, proximity is optionally determined per segment defined by each transmitter—creating a segmented proximity warning zone (segmented specification of the zone of hazardous proximity). In some embodiments, segmenting information is used to allow establishing different avoidance thresholds for different segments. For example, a segment which is more “padded” by overlying tissue of a separating wall might appear to be distant from the lumen, even though, upon insertion of a fastener in that region, there may still be a danger of trauma. In another example, a particular segment may be considered “safer” (shorter safe approach distance), e.g., because it is less likely that a fastener will be oriented in a direction which threatens it. In some embodiments, a catheter probe bends around the lumen into which it is transmitting, potentially creating non-uniformities in the spatial distribution of it signal amplitude. A segmented probe provides a potential advantage by allowing location to be more precisely identified (e.g., the contribution of more distant segments can be ignored as irrelevant to proximity detection).

Optionally, these locations are determined at an early part of the procedure, and then catheter wire 111 electrical activation is discontinued (and the catheter wire 111 optionally withdrawn). Later on, in some embodiments, an operator is warned of proximity (for example, of fasteners introduced to the area), e.g., via a displayed image (optionally, a live-updating image). Alternatively, the catheter wire 111 remains active and in place. An approaching electrode (optionally including electrically conductive device parts, which may include the fasteners) will then, in some embodiments, sense the coronary artery “warning signal” regardless of whether the relative fastener and coronary spatial positions are explicitly known. Optionally, both position mapping and proximity sensing of the coronary artery 59 are used, potentially increasing safety and/or reliability. Note that in FIGS. 2E-2G, illustration of coronary artery 59 and catheter wire 111 is suppressed for reasons of drawing clarity, but it is optionally present.

Additionally or alternatively, in some embodiments, coronary artery 59 is monitored for inadvertent punctures or near-punctures. A device (such as a fastener, e.g., a screw) which is being attached to tissue is configured to act, from its tissue-penetrating tip, as an electrode. Upon accidental penetration and/or near-penetration of coronary artery 59, a measured impedance between the device tip and a catheter wire 111 or other electrode inserted into the coronary artery will display a sudden drop. The sudden drop is an indication of a potential injury to the coronary artery (a risk of fastener penetration), and/or is detected automatically and a warning indication of the potential injury is produced. Early warning potentially leads to a reduction in the severity of accidental penetrations by allowing them to be stopped before they are made worse, and/or allowing beginning mitigation actions immediately.

In some embodiments, the spatial extent of the coronary artery 59 is mapped using measurements made by an electrode probe during insertion of the electrode probe into the coronary artery 59. The electrode probe, in some embodiments, comprises a plurality of electrodes. Different electrical fields (e.g., alternating at different radio frequencies) are induced between different sets of other electrodes; for example, body surface electrodes or electrodes on a catheter inserted into the coronary sinus. These electrical fields “tag” space through the region of the heart, including the coronary artery 59. By a process of computational reconstruction (for example, as described in WO 2019/034944 A1, entitled RECONSTRUCTION OF AN ANATOMICAL STRUCTURE FROM INTRABODY MEASUREMENTS), measurements of these “tags” at different locations can be converted from electrical measurements (e.g., of voltage and/or impedance) to measurements of spatial position. This may use constraints such as knowledge of inter-electrode distances, and assumptions about the continuity of electrical field properties as a function of position. In effect, knowing inter-electrode distances gives a measure of the mV/mm scaling at each measurement position. Constraints of electrical field continuity (alternatively described as assuming that relatively similar measurements occurred at correspondingly nearer locations) allow reconstruction to exclude solutions which meet the scaling constraint, but are too “jumpy” to be likely, or even physically plausible.

Optionally, measurements from coronary artery 59 are treated as being part of a larger set of measurements including measurements from larger regions (and from other electrode-carrying probes), for example, measurements made by a catheter-borne electrode probe moving within one or more lumens of the adjacent heart. Insofar as two or more measurement probes measure the same electrical fields (even though separated by solid tissue barriers such as heart and/or blood vessel walls), the process of reconstruction of space from a cloud of voltage measurements can use the electrical measurements of all such probes to reconstruct a common spatial model of the heart regions in which they move. The spatial positions assigned to measurements from the probe which was in the coronary artery are, accordingly, assigned to be positions of the coronary artery.

Accordingly, in some embodiments, movements of a probe in the right atrium/right ventricle (for example; it should be understood that these body cavities serve as examples of body cavities more generally) are directly assessed for their proximity to the right coronary artery (for example; it should be understood that other blood vessels are additionally or optionally mapped using this method), the position of which is modeled in the same spatial coordinate system as the space which is directly accessible to the probe. Optionally, measurements from different probes are adjusted as necessary to account for differences in electrical measurement offsets and/or gains; for example, using predetermined calibration values, and/or calibration values determined on the fly (e.g., by comparing measurements at positions known from other considerations such as limits of motion and/or landmarks to be identical and/or adjacent). It is noted in particular that inter-electrode distances used for local scaling determinations need not be the same in both electrode probes, or even the same among all electrodes.

In some embodiments, the location of coronary artery 59 is determined by another method: for example, flow of blood through the coronary artery 59 may return a Doppler signal under ultrasound imaging, or the particular dielectric properties of blood may induce an electrically sensed impedance signal for a sensing electrode approaching a portion of coronary wall behind which the coronary artery 59 lies.

In the case of electrically active structures specialized for impulse transmission, there is produced a characteristic pattern of activity which is sensed, in some embodiments of the present disclosure, by a suitably configured electrode probe when it approaches and/or contacts the structure and/or a cardiac wall within which the structure is embedded. Electrical activity of the AV node 60A and/or bundle of His 60 is optionally characterized, for example, by timing of the electrical activity relative to the body-surface recorded ECG, or by the time course of single- or dual-electrode recorded electrical activity (e.g., rise time, fall time, and/or baseline-to-baseline time).

In some embodiments, artificial stimulation (pacing) is used as part of locating electrically active structures, e.g., by noting locations from which pacing is effectively entrained, and/or by correlating measurements of electrical activity away from the pacing electrode with the known time of injection of pacing current from the pacing electrode.

In some embodiments, measurements having patterns of activity characteristic of the AV node 60A or the bundle of His 60 are located within the compound model of block 118; for example, using one of the methods of measurement coordination described hereinabove. In some embodiments, location of these structures is optionally performed as part of activities to more generally mapping the electrophysiology of the heart, for example as further described in relation to block 116.

Implantation of an annuloplasty device 112 preferably also avoids unintended interference with other structures and functions of the right atrium 51: for example, avoids blocking inflow from the coronary sinus, and avoids more generally interfering with the electrical conduction system of the heart, including nodes and pathways between the AV node 60A and the SA node.

### Valve Characterization

In some embodiments, electrically and/or electrophysiologically measured data is combined with detailed anatomical information to produce a data structure which is a compound model representing an implantation target in the heart having enough detail to support showing (e.g., on a visual display of the model) where an implantation is occurring, including a distinction between at least two of: targeted tissue (e.g., fibrous tissue for anchoring in), non-targeted tissue (e.g., cardiac muscle adjacent to the fibrous tissue), and vulnerable tissue (e.g., the coronary artery 59, bundle of His 60, and/or the AV node 60A).

To provide the anatomical information, at block 114, in some embodiments, the valve itself (e.g., tricuspid valve 57) is characterized in advance of implantation. It is emphasized that the anatomical information discussed below relates to detailed aspects of valvular anatomy. They include, in particular, shapes of the leaflets and the commissures along which they coapt (or fail to coapt), size of the valve annulus, and optionally dynamics of each. This is information which can be of particular assistance in guiding a valve annuloplasty procedure.

FIG. 4A shows some of the major structures of a tricuspid valve 57, including the three leaflets (posterior leaflet 57A, anterior leaflet 57B, and septal leaflet 57C) and the valve annulus 57D. Also shown are the AV node 60A (which should be avoided during implantation to avoid complications), and the opening to the coronary sinus 61 (inflow from which should not be physically blocked). The valve leaflets 57A, 57B, 57C come together along commissures 57E, 57F, 57G. Also indicated is a position of a portion of a coronary artery 59. What is shown is an idealized structure. Characterization of the tricuspid valve, in some embodiments, comprises obtaining information which provides more patient-specific details of valvular anatomy.

Valvular anatomy can be variable over several parameters that may affect how an annuloplasty procedure is performed. For example, there may actually be from 2-4 distinguishable leaflets (and more may be seen pathologically). The leaflets may be joined together for a longer or shorter distance before they split at their commissure, and the locations of the commissures may be different depending on leaflet number, size, and/or orientation.

A primary reason to perform annuloplasty is to reduce regurgitation consequent to the failure of valve leaflets 57A, 57B, 57C to coapt and form an adequate seal during contraction of the right ventricle 55. Different cases have different pairings of leaflets failing to coapt along their commissures. Failure to coapt may be accompanied by prolapse, where one or more of the leaflets are pushed back into the atrium, instead of correctly coapting.

Poor coaptation may be due to expansion of the valve annulus. Additionally or alternatively, there may also be holes in individual leaflets, shortened leaflets, or another morphological problem; the problem may be congenital and/or acquired. In patients with pre-existing implants, there may be interference with valve leaflet function by implant parts such as pacemaker leads. Poor coaptation potentially relates to function of the chordae and/or papillary muscles of the heart; for example, papillary muscles damaged by ischemia may fail to shorten as usual, potentially contributing to valve leaflet prolapse.

With respect to variations in anatomy and pathology, an important issue for planning is how to attach a valve annuloplasty device so that it operates to good results together with the patient's specific anatomy, consistent with avoiding damage to vulnerable structures, for example, those identified by electrical and/or electrophysiological techniques (e.g., as described in relation to block 101).

When annuloplasty device 112 is cinched (or otherwise actuated), for example, it is preferable, in some embodiments, that the annuloplasty device 112 exert shrinkage upon portions of the valve 57 which are more likely to benefit from this treatment—e.g., annulus shrinkage may be more preferable through a circumferential extent crossing the posterior-anterior commissure, or crossing the anterior-septal commissure. Since the posterior-anterior commissure is circumferentially further from sensitive structures specialized for impulse transmission such as the AV node 60A, the difference in emphasis on where treatment is critical may also relax constraints on the placement of fasteners, potentially making it easier to avoid sensitive structures.

Valve annulus expansion has been found to be greatest on portions of the valve annulus circumference away from the septal wall. Some annuloplasty devices 112 have two free ends (for example as shown in FIG. 6). It may be preferred for a particular procedure to avoid placing these free ends on opposing sides of a commissure—or if the coaptation at the commissure is normal, such placement may, on the contrary, be preferable (for example, to avoid over-closure of the valve 57

Furthermore, the valve 57 by its nature is not a static structure, and may be recorded dynamically as it changes shape over the course of a heartbeat cycle and/or over the course of a respiratory cycle. Functional information, in some embodiments, is extracted from valve behavior over time. The shape of the tricuspid valve 57 during right ventricle 55 contraction (when the tricuspid valve 57 should be closed) is particularly useful as an indication of regurgitation. Additionally or alternatively, the valve's open and/or transitional states can be used in determining aspects of valve anatomy such as commissure position and/or length. Transitional states of valves may also reveal information about the functional state of the valve 57, for example revealed in the by time course of valve leaflet motions and/or leaflet “flutter”. Transitional state motions characteristic of the valve leaflets may also help to identify them, for example, when performing component analysis to isolate cyclic components, e.g., as described in relation to FIG. 7B.

The region of valve annulus 57D itself may show a mix of behaviors as a function of heartbeat phase, e.g., different for muscular parts (which will expand and contract during the heartbeat cycle), and fibrous tissue of the valve annulus 57D itself (which is relatively static, but may still be affected by muscular forces acting on it). The distinction of fibrous and muscular tissue may be difficult to make purely from motion analysis, however; and in some embodiments is confirmed by, determined instead by, or determined jointly with the use of electrophysiological measurements, for example as described in relation to block 116. Tension in the valve annulus 57D is potentially indicated by a degree of flexibility shown during the heartbeat cycle; the tension in turn provides, for example, a potential indication of where tightening of the valve ring is more likely to have intended treatment effects.

During implantation, it is preferable, in some embodiments, to anchor in the fibrous tissue, since it is more stable; and furthermore, there is perceived to be a lower likelihood of inducing functional damage. However, the ring of fibrous tissue can be thin, increasing a need for high-resolution anatomical information and/or electrophysiological confirmation.

The valve annulus 57D may undergo phasic changes as a function of respiratory phase and/or mode of ventilation. Positive pressure ventilation may tend to compress the heart (with corresponding effects on the valve annulus circumference), potentially complicating the annuloplasty decision as to how much cinching is needed so that the valve functions correctly under normal respiration pressures. Normal respiration can also produce phasic changes in heart morphology which potentially include alterations in valve annulus 57D size.

In some embodiments, phasic movements (changes in shape) are accounted for by methods of frequency-correlated component analysis, described, for example, in relation to FIG. 7B.

Methods of obtaining three-dimensional images and/or functional images of heart valves— potentially including resolution of locations of insufficient coaptation—include ultrasound and electrical field-based imaging (measurement) methods, used in some embodiments of the present disclosure. Ultrasound-based methods include transthoracic echocardiography (TTE), intracardiac echocardiography (ICE), transesophageal electrocardiography (TEE), and tissue Doppler echocardiography (TDE). Electrical field-based imaging methods, in some embodiments, comprise moving an electrode probe within a cardiac chamber while making electrical measurements of exogenously produced electrical fields—and reconstructing the shape of the cardiac chamber using the measurements and optionally additional information such as relative spacings of the measurement electrodes, and/or prior knowledge of aspects of the electrical field's spatial distribution. In some embodiments, electrical measurements are made using electrodes placed on either side of the valve 57.

Electrical field-based measurements can also be used to sense regurgitation (for example, in analogy to information on blood flow patterns produced using TDE), by a method comprising injecting and/or tagging fluid to create tracer fluid in a ventricle, and measuring disturbances in magnetic and/or electrical properties caused by retrograde migration of the tracer fluid through a regurgitating valve. The tracer fluid can comprise anything which creates an electrical, dielectric, and/or magnetic contrast; for example by injection (e.g. of saline or another fluid), temperature manipulation (e.g., localized heating of blood), and/or structural manipulation (e.g., using ultrasound to create cavitations in blood). The disturbance measured can be, for example, changed voltage or magnetic field strength measured from an induced electrical field and/or magnetic field, or changed impedance measured by an electrode. Additionally or alternatively, FIG. 2C illustrates the placement of a pigtail catheter 113 in the right ventricle, from which location boluses of saline or another tracer fluid are optionally injected. In cases of regurgitation, the tracer may end up leaking across the heart valve 57 where it affects the electrical environment of the right atrium 51, and sensing, e.g., by multi-electrode probe 102.

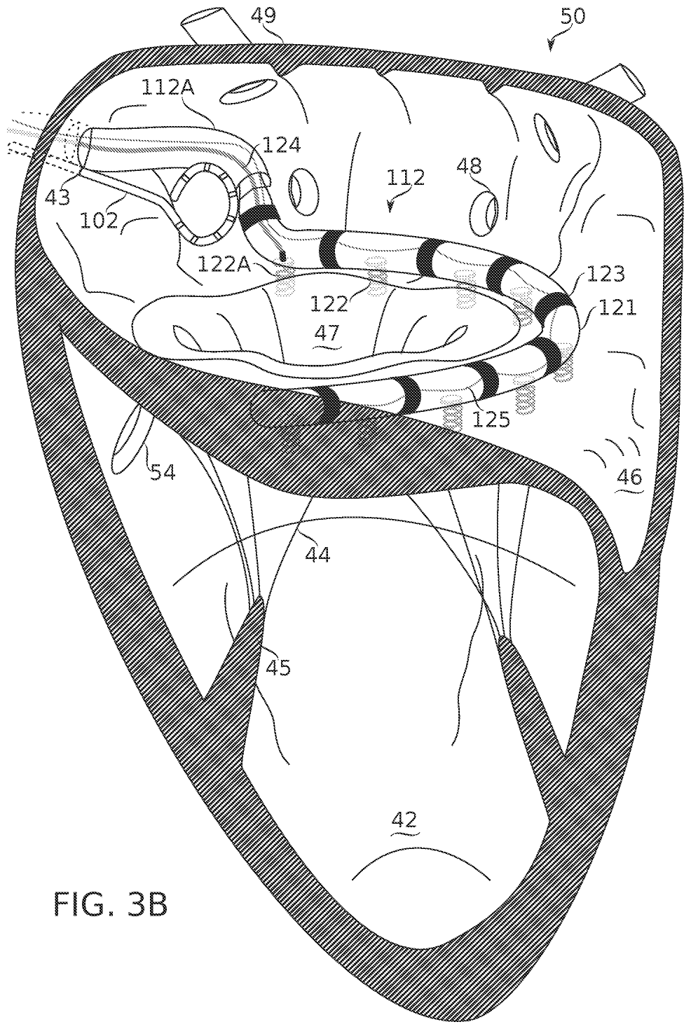

Brief reference is made to FIG. 5A, which schematically represents a graph of impedance changes as a function of time (in seconds) as measured between electrode pairs of a multi-electrode probe 102 (e.g., a lasso-type catheter) positioned in the right atrium and generally “above” the tricuspid valve leaflets along an atrioventricular axis, according to some embodiments of the present disclosure. Herein, reference to “impedance measurements” should be understood to include measurements of electrical signals which change as a function of impedance change, and/or are indicative of impedance and/or impedance changes. The electrical signal can be sensed, for example, via one or more electrical circuit properties such as voltage, current, resistance, and/or reactance. For example, impedance measurement is optionally performed by measuring voltage changes in a constant-current condition, and converted to impedance using electrical circuit analysis techniques (with body tissue and/or fluid as part of the circuit). Additionally or alternatively, voltage measurements are used directly as a proxy for impedance changes. Resistance and electrical current measurements are also optionally used additionally or alternatively to impedance measurements as such.

As a leaflet moves nearer to an electrode pair, its relatively insulating properties (e.g., compared to blood) lead to an increase in measured impedance between them; as it moves away, impedance drops again. Onsets of opening and closing events are noted on the graph as open circles (onset of opening), filled triangles (onset of a transient partial closing), filled circles (onset of re-opening), and open triangles (onset of full closure). This pattern repeats with the repeating heart cycle. Its features, seen using impedance measurements, are also characteristic of motions of the heart valve seen, for example, in an M-mode (motion mode) ultrasound recording.

Impedance changes with a timecourse like that shown in FIG. 5A may also be measured by an electrode brought into close proximity to, and optionally intermittent contact with, leaflets of the valve. A characteristic feature of valve motion, in some embodiments of the present disclosure, is the occurrence of a doubled opening/closing cycle over the course of a single heartbeat cycle. In comparison, motion of connective tissue of the valve annulus itself, for example (e.g., fibrous tissue region comprising annulus 57D of FIG. 4A), displays little or no cycle doubling.

CT scans and MRI scans are also potential sources of anatomical information. Apart from structural information, magnetic resonance imaging, can optionally provide a measure of regurgitation, for example, based on stroke volume differences between the left and right ventricles.

It should be understood that early measurements indicating valve (and valve leaflet) anatomy may have a relatively low resolution limited by the low number of measurements available. As more measurements are obtained and/or are more computer processing time is applied to producing the compound model, available image detail may correspondingly increase.

### Endogenous Electrophysiology Measurements

At block 116, in some embodiments, endogenous electrophysiology is mapped. Electrophysiological mapping comprises moving an electrode probe within the heart to visit various positions, concurrently with measuring endogenous electrical activity at the visited positions.

In some embodiments of the present disclosure, movement of the electrode within the heart is accompanied by measurements using a second modality. The measurements using the second modality serve as scaffold measurements (indicating spatial locations) for the compound model, and/or supplement the electrophysiological mapping measurements in generating the scaffolding of the compound model. For example, the same electrode probe may also be used to make measurements of exogenously generated electrical fields extending through the heart chambers, and the resulting measurement used to generate a position map of the heart. In another example, ultrasound images showing a position of the probe are correlated with electrophysiological measurements made using the same probe.

The electrophysiological measurements, in some embodiments, are presented in image form, mapped to positions defined by the compound model. This can be done in different ways, and optionally more than one within a single image. In some embodiments, for example, color coding is used to distinguish times at which the heartbeat impulse reaches different areas, earlier or later in the heartbeat cycle. In some embodiments, waveforms with different characteristics (e.g., relative amplitudes of components) are assigned to different categories, and shown differently in the image in accordance with category—for example, areas with waveforms characteristic of proximity to structures such as the AV node and the bundle of His are tagged by a visual marker, and/or shown differently (e.g., a different color, brightness, saturation, transparency, and/or texture) than areas with waveforms characteristic of cardiac tissue alone.

In some embodiments, a certain ratio of P wave amplitude to QRS complex amplitude (e.g., a ratio of 1:1.5, 1:2, or another ratio) is assigned as indicating a position at the valve, and “midway” along an atrioventricular axis. Optionally, positions at the valve are indicated by a range of ratios, for example, 1: (1.5±0.25), 1: (2±0.25), or another range. Locations at which a larger ratio P wave to QRS complex amplitude is measured are assigned as “atrial”; locations where a relatively smaller ratio is measured are assigned as “ventricular”. It is noted that the valve annulus comprises connective tissue which presents a barrier to the propagation of waveforms between the atrium and ventricle, helping to sharpen the spatial transition between P wave-dominated and QRS complex-dominated regions. Optionally region through which the transition occurs is identified as comprising positions at the valve. In some embodiments, gradations between two different characteristic waveforms are color coded according to threshold values, and/or shown along a color or other visually displayed gradient. For example, conversion along an atrioventricular axis from a dominant (higher-amplitude) P-wave 504 to a dominant QRS complex 503 (FIG. 5B) is shown by different colors selected from a color look-up table.

By making repeated measurements at different locations (e.g., different radial offsets from an atrioventricular axis extending through the center of the valve annulus) an orientation of the valve annulus can also potentially be revealed. For example, establishing the positions—in a spatial frame of reference—of three locations which are at the level of the valve annulus may be used to define a plane which intersects the valve annulus at two opposite sides of the valve annulus circumference, and optionally all around its circumference. This allows estimation of the orientation of the valve annulus, as well as its atrioventricular axis location. The reference positions need not all be directly measured locations at the valve annulus level. For example, a reference function indicating rates of transition between atrial-side and ventricular-side electrophysiological signals as a function of movement along the atrioventricular axis can be measured during a single passage between the two sides. The valve-level positions can then be estimated using the same transition function to interpolate between measurements made above (atrially) and below (ventricularly) the level of the valve annulus. A potential advantage of having an estimate of the orientation of the valve is in planning, tracking and/or evaluating movements of a probe involved in structural heart disease treatment of the annulus as it moves around a significant portion (e.g., at least half) of its circumference, e.g., as may be performed during implantation of an annuloplasty device. In some embodiments, the identification of the valve annulus location comprises identifying a plane that is estimated to intersect its full circumference. In case a valve annulus is distorted to a non-planar shape (e.g., saddle-shaped, for example due to a geometric deformity of the valve annulus), measurements at a plurality of locations around the valve annulus may determine this by sensing different atrioventricular axis displacements at different circumferential locations.

In some embodiments, a full waveform (covering a full heartbeat cycle) is assigned to a single “position” as defined by the scaffolding coordinate system of the compound model, for example a time-weighted average of positions measured during the full heartbeat cycle. In some embodiments, measurements of partial waveforms are assigned to the exact compound model-defined location of measurement (with a precision insofar as is known). Optionally, a full waveform for a particular position at which a full waveform is constructed using data from neighboring positions. For example, time points of the cyclic waveform with missing data receive information from a nearest neighbor, by (e.g., distance-weighted) averaging of nearby model positions for which measurement data is available, and/or by another method of interpolation. Additionally or alternatively, interpolation of missing full cycle data may also be performed between times for which data is available. In some embodiments, interpolation is jointly performed over time and space; e.g., the shape of the nearest available (in position space) waveform portion for a period of a heartbeat cycle is amplitude-scaled to fit the available measurements at a particular position which overlap in time.

Information acquired by electrophysiological mapping measurements, in some embodiments, has been described in part with reference to block 101 (localization of structures such as the bundle of His 60 and the AV node 60A), and to block 114 (electrophysiological distinguishing of fibrous valve annulus tissue and myocardial tissue). Those electrophysiological mapping operations may also be considered to fall within the scope of the operations of block 116.

In some embodiments, electrophysiological mapping comprises measuring differences in electrophysiological properties in a way that correlates electrophysiological signals with spatial position along one or more axes (e.g., along an atrioventricular axis), and/or distinguishes different positions as being, for example, atrial, within the valve (atrioventricular), or ventricular. For example, atrially-measured electrical activation tends to have a relatively high amplitude at times corresponding to the P-wave 504 of the normal, externally recorded ECG. Ventricularly measured electrical activity corresponds to a higher amplitude at the time of the QRS complex 503. Positions in between (at the level of the valve itself) tend to be intermediate in character. They may also show waveforms corresponding to other signals, for example, at locations in proximity to the bundle of His 60.

Endogenous electrophysiological signals propagate with characteristic propagation velocities, so that changing latency may also serve as a marker of probe position. Moreover, different components of endogenous electrical activity may have different conduction velocities; for example, signals propagate faster through the AV node and bundle of His than through other myocardial cells. Optionally, differences between the phase (optionally measured, for example, by time-to-onset, time-to-peak, or another metric) of two different electrical signal components provide an indication of probe position—for example, an electrical impulse propagating generally in a ventricular direction along an atrioventricular axis arrives at an atrial position before arriving at a ventricular position or a valvular position.

Additionally or alternatively, electrophysiological mapping potentially distinguishes between types of tissue: myocardial or fibrous, for example; and/or locations of impulse transmission-specialized structures such as the AV node 60A and the bundle of His 60. Fibrous tissue is non-contractile, so electrical signals measured while in contact with it are, for example, dampened compared to nearby myocardial tissue. This is used, in some embodiments, to assist in identification of the bounds of fibrous tissue in the valve annulus.

### Integration of Information in a Compound Model

At block 118, in some embodiments, measurement data from any of blocks 110, 101, 114, and 116 is integrated into a compound model.

In some embodiments, the position map of block 110 is used as the scaffold for the compound model. In embodiments where it is used as the model's “scaffold”, the position map provides a unifying frame of reference, to and/or through which the measurements of other measurement modalities are related as being, for example “closer” or “further” from one another. This may be useful during a procedure, for example to assist in finding, approaching and/or avoiding certain targets.

In the case of a position map, distances can be expressed in terms of physical space (spatial distance), but other types of distances (examples are mentioned below) may also serve for purposes of procedure guidance and/or monitoring.

Once association to position map locations is established for a measurement (different methods of doing so are described, for example, in relation to blocks 101, 114, and/or 116), it is integrated, in some embodiments, into a compound model of heart function and anatomy which can be used for display, for providing procedure guidance, and/or for monitoring one or more aspects of procedure status. In some embodiments, the display shows both the compound model of the heart and the estimated locations within the model of equipment introduced to the heart; for example, positions of measurement probes and/or the annuloplasty device.

It should be understood that the compound model, in some embodiments, comprises a data structure which models the heart, and is not necessarily a visible production such as an image for display (though it may, in some embodiments, be an image and/or be used to generate an image; including, in some embodiments, a live-updated image that shows changes in positions and/or shapes of heart structures and/or equipment introduced to the heart as a procedure is carried out).

The data structure is not necessarily representative of physical space (for example, it may be representative of a “measurement space”), though representation of physical space is a feature of some embodiments of the present disclosure. It is convenient and, in some embodiments, preferable for the “scaffolding” which unifies data of different types to be expressed in terms of spatial position; for example, since this is readily represented and understood by a surgeon performing a procedure. However, e.g., if the operations of block 110 are omitted, then the “scaffolding” provided by a position map is optionally provided instead by combining data from one or more of the operations of blocks 112, 114, and/or 116, and may not include spatial position data as such—but rather represent a kind of “functional topography”, wherein distortion of absolute distances is allowed, and procedure-relevant characteristics of the mapped region are more heavily emphasized by features of the display (such characteristics optionally include, for example: “on the fibrous tissue of the valve annulus”, and/or “dangerously close to the bundle of His”).

The resolution of the compound model (and/or images generated therefrom), in some embodiments, is adaptive to the available data. For example, a “rolling ball” algorithm is used, in some embodiments to generate a connected surface from a cloud of measurement positions. The algorithm behaves, in some embodiments, as if a ball of a certain diameter is brought from outside the cloud into contact with it, as close to the cloud center as contact with the cloud measurement positions permits. Optionally, the algorithm includes refinements such as a noise-reducing “elasticity” parameter allowing the ball to penetrate a short distance past single measurement positions, but for a shorter distance past a plurality of measurement positions. In some embodiments, the parameters of the rolling ball are changed (e.g., the diameter is decreased) as more measurements become available, and optionally changed differently for different parts of the measurement cloud according to measurement density.

A principle which may be used in some embodiments of the present disclosure to enable combination of data from different measurement modalities to a compound model is that of coordinated measurement: measurements made in different modalities which can be determined to have been made “at the same location” may thereby be integrated into a single compound model as belonging to a same location. Measurements at known offsets in time or space may similarly be related to one another and integrated into a compound model at different locations with corresponding offsets. Moreover, coordinated measurements—e.g., because they occur close in time and/or space—may provide anchoring to other measurements of the same modality (even measurements not directly coordinated with measurements of another modality), allowing the compound model to be formed with greater detail.

For example: while an electrode is used to map endogenous electrophysiology (discussed further in relation to block 116), 2-D X-ray or ultrasound imaging (e.g., as mentioned in relation to block 114) may be used to note the probe's relative position (at least in part) at the moment of some or all electrophysiology measurements. For example, position may be measured in one plane, or otherwise constrained, even if not fully determined. While this information may be insufficient to reconstruct a surface or volume suitable for a position map, the coordinated measurements may nevertheless serve, in effect, to allow partial relative positions (e.g., coordinates within a projection plane) to be determined. In some embodiments, this plane serves as “scaffolding”, allowing at least partial determinations of relative distances along certain spatial directions.

In some embodiments, none of the coordinated data measurement methods measures spatial position as such. For example, locations of anatomical structures of special concern (described in relation to block 101), are optionally characterized, e.g., by their electrical impedance, and/or sensitivity to exogenous manipulations such as fluid injection or pacing. Similarly, endogenous electrophysiology activity measurements may be characterized, at particular locations, by a characteristic linear or non-linear combination of ECG wave components such as the P wave 504, QRS wave 503, and/or waves associated with localized structures such as the AV node 60A (buried within the septal wall) and/or the bundle of His 60. Though neither of these measurements is spatial in nature, they can still be used to distinguish locations at which different measurements are made.

It can, furthermore, be determined by one or more of various means (according to the particular embodiment), that two measurements in two different measurement modalities (e.g., impedance and electrophysiological measurements) are coordinate: for example because they are measured using the same probe at the same time (optionally with the same or different electrodes/sensors), because they are measured in same observed positions under imaging by a third modality (whether or not that position is itself characterized as having particular spatial coordinates), because they are measured by two probes in physical contact or otherwise physically constrained to be near one another, or by use of another constraint.

Where coordination of two measurement modalities is not inherent and/or constant, there may still, in some embodiments, be available a limited set of measurements that can be considered coordinate; e.g., because they are known to have been made at the limit of movements of a catheter probe, during travel along similar paths, or for another reason.

Furthermore, for purposes of setting up non-spatial coordination between different measurement modalities, “distances” may be calculated as existing along one or more non-spatial axes related to measurement characteristics—for example, signal phase, amplitude, or one or more eigenvalue vector components. Insofar as measurements of physical values tend to change continuously over time and/or space, measurement distances may be used as information to help establish that measurements are coordinate, and from this a corresponding compound model incorporating those measurements may be established.

Non-spatial metric distances may optionally be used as part of guidance and/or monitoring. For example, in some embodiments, a certain physical distance constraint such as “no fasteners placed within 4 mm of the AV node” is to be satisfied. This constraint is optionally considered to be functionally met by a non-spatial constraint related to one or more non-spatial measurements. For example, in an electrophysiological signal, a relative amplitude and/or timing of a wave component may be required to be uncharacteristic of locations nearby the AV node 60A: for example, peaking at least 3, 5, 10 or more msec too late, and/or at least 10, 20, 30 or more mV different in amplitude.

### Planning and Tracking of the Implantation Procedure

At block 120, in some embodiments, the implantation procedure is performed, comprising within it stages of final planning and implantation itself.

### Multimodal Measurement-Revealed Contraindications

Optionally, the annuloplasty procedure is abandoned at this stage, due to a discovery of one or more contraindications. For example, it may be determined during a procedure that the valve leaflets themselves are damaged or malformed in a way (e.g., with holes, torn, shortened, and/or by fusion with portions of a previously implanted device) that makes the annuloplasty device unlikely to produce beneficial results. In some embodiments, it may be determined, based on inspection of the compound model, that no acceptable surgical solution is available, for example due to proximity of vulnerable structures to the planned location of annuloplasty device implantation, lack of stable tissue for implantation, or another reason.

### Multimodal Measurement-Guided Procedure Planning

Assuming the procedure continues, criteria already outlined above along with additional relevant criteria can now be applied to selecting a specific targeted configuration of the annuloplasty device as it attaches on the valve annulus.

Brief reference is now made to FIGS. 4B-4C, which schematically illustrate examples of displays used in device implantation planning and/or in performing device implantation, according to some embodiments of the present disclosure. FIG. 4B represents an above-the-plane view of a simplified representation of a tricuspid valve, while FIG. 4C represents a cutaway transverse view of a simplified representation of a tricuspid valve.

In some embodiments, a hinge of the valve (that is, the approximately circumferential region wherein the valve annulus ring gives way to the valve leaflets) is identified by any suitable mix of manual and automatic identification methods. Identification of tricuspid valve features including the valve's hinge is described, for example, in relation to FIG. 7A. The identification is optionally completely automatic. For example, any one or more of hinge “cliff” geometry, position along an atrioventricular axis and local impedance values are used and/or combined to identify the internal boundary of the valve annulus ring. Optionally, any one or more of these indications are shown to a user, and the user identifies the hinge location. Optionally, an automatic determination is shown to a user and the user is allowed to correct the automatic determination.

In FIGS. 4B-4C, path 403 represents an estimated circumferential location of a valve annulus hinge; drawn by a user and/or automatically determined. Optionally, locations of other structures are identified in the views of FIGS. 4B-4C. for example, optional markers 404, 405 represent approximate positions of an opening into the coronary sinus and the bundle of His, respectively. An estimated position of the right coronary artery is shown along path 401 (FIG. 4B) and/or a spatial representation of the coronary artery 401A (FIG. 4C). Path 402 represents a planned path along which an annuloplasty device is to be implanted. In some embodiments, path 402 is automatically calculated, taking into account the estimated positions of the coronary artery and the hinge of the valve annulus ring, e.g., expanded radially outward about 3 mm from path 403; optionally adjusted to maintain a minimal clearance from the estimated position of the right coronary artery; e.g., a clearance of 3 mm.

The two views of FIGS. 4B-4C are optionally shown simultaneously or alternately. FIG. 4B is optionally shown during planning, in particular, to allow a clear view of structures along which the annuloplasty device is to extend. The transverse view of FIG. 4C also has potential advantages during device implantation, for example as described hereinbelow.

An annuloplasty device is selected to match the shape and size of the patient's tricuspid valve, and/or the nature of tricuspid valve coaptation problems found; as measured, for example, in block 114. In some embodiments, an annuloplasty device which is otherwise-than-optimal for the size/disease is optionally selected in view of safety criteria (i.e., to ensure that vulnerable structures are avoided); for example to provide extra security that the device can be secured into place without damaging a vulnerable structure.

For procedures where the annuloplasty treatment target is to shrink the valve annulus, a target final size of the valve annulus is also selected. In some embodiments, the target size is simply selected to restore a valve diameter, circumference, and/or radius which is considered standard for healthy patients of the current patient's, e.g., size and/or age. Optionally, this is adjusted for the state of the valves themselves; for example, a valve may receive extra tightening for a patient with a particularly large regurgitating aperture, or a clear propensity for valve prolapse. It should be verified that a resizing annuloplasty device can be shrunk (e.g., cinched) enough to reach the target diameter. Alternatively or additionally, an annuloplasty device is provided for bracing; i.e., stiffening of the valve annulus, which potentially helps to enhance coaptation and/or resist deformations which can lead to valve regurgitation.

Optionally, sizes are selected while compensating for the physiological conditions of the implantation procedure. For example, if positive pressure ventilation is used, the valve annulus may appear smaller than it normally is. Insofar as a certain amount of elasticity may be retained even after shrinking of the annuloplasty device, it may be that the appropriate intervention should involve somewhat more tightening than the level of regurgitation measured under ventilation conditions indicates.

In some embodiments, planning also includes selection of a starting point of the implantation, and selection of the placement of fasteners. This potentially involves finding an implantation solution which simultaneously satisfies a number of criteria:

Other things being equal, the annuloplasty device itself should span the leaflets and their commissures in a way that is best-suited to close up gaps, and without tightening being misdirected to leaflet regions that might not benefit (this is discussed, for example, in relation to block 114).

Fasteners (e.g., screws) are preferably placed, in some embodiments, roughly centered on either side of commissures to be tightened, rather than at the radial position of the commissure itself.

Ends of the annuloplasty device (for an open-loop design) may preferably be placed near edges of the septal leaflet, limiting the span of the device which extends along the septal wall, where some vulnerable structures like the bundle of His and AV node are (the septal circumference is also said to be the segment of the valve circumference which is often in least need of tightening).

The fasteners of the annuloplasty device should be placed close enough together to properly secure the device, e.g., without loose ends, without a tendency to form arches/loops between fasteners, and circumferentially close enough to each other to avoid drawing the annuloplasty device across the valve aperture when tightened, partially blocking it.

The fasteners, however, should not be placed where there is a significant risk of damaging a vulnerable structure, for example the AV node, bundle of His, and/or the coronary artery 59. Optionally, fastener spacing is adjusted wider or narrower to increase the available margin of error with respect to vulnerable structures. Optionally, fasteners are planned to be inserted at irregular intervals to increase the available margin of error at specific locations.

Variability associated with fastener positioning is optionally accounted for. For fastening operations entailing a higher risk of variability, it is optionally preferred to plan to avoid by a greater distance locations of vulnerable structures. For example, the first fastener is potentially more prone to positioning error, since it cannot depend on any previous fastener to help control slippage. It may optionally be planned to place fasteners out of order along the circumference of the annuloplasty implant, potentially transferring variability away from a certain location of particular risk.

In some embodiments, a model of an annuloplasty device selected for use (or a candidate for use) is placed within the context of the compound model (together with its fasteners), and the placement evaluated for how well it satisfies a plurality of different criteria. For example, at least one criterion of safety (e.g., margin of distance of fasteners from vulnerable structures) is to evaluated, and at least one criterion of functional efficacy (e.g., anticipated degree of post-operative regurgitation) is evaluated. In some embodiments, the criteria are automatically evaluated for a number of different arrangements of the annuloplasty device and its fasteners (the tested arrangements themselves may also be automatically selected, for example by systematically and/or randomly adjusting positions of device portions). In some embodiments, evaluation is carried out automatically at least in part: for example, distances between fasteners and vulnerable structures are measured automatically using distances represented the compound model/annuloplasty device model; and/or post-operative regurgitation is estimated by shortening a modeled circumference of the valve while assuming valve leaflets retain their already-measured area within that shortened circumference. Optionally, evaluation uses a heuristic, for example, an estimate of risk and/or benefit based on statistically correlated outcomes of past procedures with similar characteristics.

In some embodiments, one or more of the most optimal of the arrangements evaluated is presented (e.g., in a projection or other display of a 3-D view) to an operator for review, manual adjustment, and/or final selection.

### Multimodal Measurement-Guided Annuloplasty

FIG. 2D shows multi-electrode probe 102 retracted to hover above valve 57, from which location it is used, in some embodiments, to monitor (electrically image) the valve during the implantation procedure. Additionally or alternatively, another imaging monitoring probe is used, for example as described for positioning measurements in relation to block 110.

FIG. 2E shows annuloplasty device 112 at the beginning of the implantation procedure, having been advanced into the right atrium over catheter 112A, partially extended from catheter 112A, and partially attached by (e.g., two) fasteners 122. In some embodiments, fasteners are attached by use of a fastener-attached control member 124 operating through catheter 112A and from inside sleeve 121, for example as shown in FIG. 3B (which shows implantation of a mitral valve annuloplasty device using the same working principle as described for the tricuspid valve annuloplasty device 112).

Placement of fasteners, in some embodiments, is guided at least in part by data acquired using the fastener 122 (and/or a position-associated structure such as the fastener's placement control member 124 and/or delivering catheter sheath 112A) itself as an active and/or sensing element. In some embodiments, fastener 122 comprises a metal portion, configured for use as an electrode, for example by attachment to control member 124 (which may itself be conductive), and via that attachment attached to an electrical measurement device (e.g., a voltmeter outside the body).

Herein, a “position-associated” element to, e.g., a fastener 122 is an element (comprising a radiopaque marker, for example, and/or an electrode) which, while not part of the fastener 122 itself, is nevertheless linked to it by a regular and/or predictable offset in position, so that knowing the position of the position-associated element allows estimation of the position of the fastener 122 itself. Optionally, the position estimate uses further information, such as a relative distance of advance into the heart of the position-associated element and the fastener 122.

There are several types of measurements (each comprising a different measurement modality) which may be relevant to position finding, optionally used together in any suitable combination. Examples include the following.

In some embodiments, voltages sensed through fastener 122 (and/or, optionally, a position-associated electrode; for example, an electrode of a catheter sheath 112A used to deliver fastener 122, and/or an electrode of control member 124) are matched to positions in the position map of block 110, through being measurements of same voltage fields which were used in measurements that generated that position map in the first place.

In some embodiments, placement of fastener 122, e.g., along an atrial-ventricular axis, is guided by measuring endogenous electrical signals using fastener 122 (or another position-associated electrode) as an electrode. For example, the targeted position of the fastener comprises a position at which endogenous electrical signals match an atrioventricular waveform (preferably while avoiding a waveform that also shows evidence of the AV node being nearby). Optionally, a graphical (e.g., 3-D) representation of fastener 122 is dynamically adjusted to represent its position along an atrioventricular axis, for example, presented with a color or other surface characteristic indicative of “atrial”, “ventricular” and or “valve annulus” positioning of the fastener 122.

In some embodiments, depth of insertion of a fastener 122 is determined by measuring how voltage, current and/or impedance sensed from it (and/or injected through it) changes as more of it becomes embedded in tissue.

In some embodiments, an angle of fastener 122 is determined, e.g., by making electrical measurement concurrently with injecting current through it. Electrodes located, e.g., at the position of multi-electrode catheter probe 102 in FIG. 2D “see” a different disturbance in voltage depending on the direction in which fastener 122 is pointed. It may be noted that a preferred direction of insertion of fastener 122, in some embodiments, is pointed directly away from the position of multi-electrode catheter probe 102, an orientation at which fastener 122 will potentially appear most “point like”. At a perpendicular orientation, for example, fastener 122 potentially appears as a more laterally-extended current source from the perspective of probe 102.

In some embodiments, placement of a fastener 122 in an intended tissue type is determined, e.g., by confirming (optionally, before placement is finalized by attachment) that impedance measurements match the expected impedance of the targeted tissue, and/or confirming that endogenously produced electrical signals from the heart measured from the fastener 122 (and/or another fastener position-associated electrode) do not indicate placement within myocardial tissue, and/or do not indicate insertion to a vulnerable location such as the AV node. Optionally, pacing signals are transmitted from fastener 122, e.g., to help confirm that it is not (because of a failure of pacing entrainment) in the vicinity of a vulnerable structure. Optionally, pacing signals are delivered from another heart location, and relative timing of pacing signal delivery and sensing from fastener 122 (or position-associated electrode) used to confirm an intended location of fastener 122, e.g., based on the latency and/or signal strength.

It is noted that the electrical connection between control member 124 and an external electrical signal measuring device may itself form a transducer; e.g., as control member 124 rotates fastener 122, control member 124 may also, in some embodiments, rotate within the grip of a clip (e.g., an alligator clip) that attaches it to the signal measurement device. This may result in small changes to contact quality in a pattern that tracks the rotation, allowing estimation of insertion depth of a screw-type (rotatingly inserted, whether with an external thread, formed as a coil, or otherwise shaped for helical advancing) fastener.

In some embodiments, fastener 122 is used as a transmitting device. In some embodiments, the transmitting is electrical, and sensed, e.g., by multi-electrode catheter probe 102. In some embodiments, fastener 122 is vibrated (e.g., using a vibration crystal, optionally in contact with fastener 122, and/or indirectly through vibration of control member 124), and the transmitted vibrations sensed (e.g., using an ultrasound sensor) to determine one or more aspects of placing the fastener—e.g., distance from one or more sensors (determined, for example, from the relative phase of vibration excitation and vibration sensing), and/or contact with tissue (determined, for example, by vibration damping and/or transmission of vibration through an alternative pathway).

In some embodiments, an imaging modality such as X-ray and/or ultrasound is used, constantly or intermittently, to help register and/or confirm the locations of measurements made in other measurement modalities.

Optionally, prior constraints on the size, shape, and/or position of the fastener are used to limit interpretation of location based on multimodal measurements—for example, if the implantation procedure has begun, then the anchored portion of the annuloplasty device 112 is optionally assumed to limit plausible fastener 122 locations to be between the catheter 112A and the last-implanted fastener 122. In some embodiments, a position-associated electrode (or other marker, e.g. a radiopaque marker when X-ray imaging is used) located on catheter 112A allows its position to be determined. Advance of control member 124 is optionally measured (e.g., via a transducer) past a position of initial exposure of fastener 122 to the internal electrical environment of the heart, and the combination of catheter 112A position and control member 124 advance used to estimate a current location of fastener 122. Optionally, annuloplasty device 112 itself comprises dielectrically distinct structures such as radiopaque markers 123. Effects of radiopaque markers 123 on nearby electrical fields potentially cause measurement changes that indicate, in some embodiments, when a fastener 122 approaches and/or passes a certain radiopaque marker 123.

A potential advantage of using a plurality of measurement modalities for tracking locations of fasteners 122 is to reduce situations of position tracking ambiguity. For example, if one measurement modality alone is consistent with a plurality of different positions, and/or prone to measurement noise that increases uncertainty, conjoining it to a second measurement modality may resolve some ambiguities.

From the perspective of validation (during the implantation phase of the procedure): if successful, placement of a fastener 122 comprises, in some embodiments, satisfaction of a plurality of criteria. It is a potential advantage to demonstrate this by a corresponding plurality of measurement modalities. For example, the available measurement modality data jointly confirm, in some embodiments, any suitable (and optionally changing according to the stage of the procedure) combination of the following specifics, which are given as examples:

The fastener is at targeted spatial coordinates.

The fastener is at a targeted location along the atrioventricular axis consistent with locations at the level of the valve annulus.

The fastener is not in contact with myocardial tissue.

The fastener is in physical contact with fibrous tissue consistent with electrophysiological and/or impedance properties of the valve annulus.

The fastener is not in intermittent physical contact with tissue (which might indicate location at a moving valve leaflet).

The fastener is not in the vicinity of one or more vulnerable structures with a characteristic electrophysiological signature (such as the AV node).

The fastener is not in the vicinity of one or more selected “tagged” structures, such as a coronary artery 59 marked by a transmitting catheter wire inserted thereto.

The fastener is not in the vicinity of one or more selected mapped structures; such as a bundle of His, or a coronary artery 59 having positions mapped using measurements of electrical fields using electrodes of an electrode catheter inserted thereto.

The fastener is extruded from the catheter (e.g., not shielded by electrically insulating properties of the catheter).

The fastener is oriented as planned for attachment (e.g., attachment by rotation to screw into tissue).

The fastener is tissue-inserted to a planned depth (e.g., has an expected increase in effective impedance due to be being partially embedded in tissue).

It should be noted that some combinations do not require a specific determination of a location's spatial coordinates.

Display of Multimodal Measurements and/or the Compound Model

In some embodiments, the compound model is rendered as a 3-D image (and/or projection of a 3-D image), and the location of features estimated from one or more of the multimodal measurements shown on the 3-D image by markings; for example, differences in the appearance of surfaces represented by the compound model, and/or markers such as symbols or shapes placed alongside surfaces represented by the compound model. Descriptions of different ways of displaying electrophysiological measurements on an image generated from a compound model are described, for example, in relation to block 116 of FIG. 1.

Optionally, an X-ray and/or ultrasound image (static or live-updating) is used as a background onto which positions and movements of other procedure elements (e.g., catheters, probes, and/or the annuloplasty device 112 itself) are projected during the procedure. Projection optionally includes aspects of the anatomical structure; for example, a 3-D image of a valve may be overlaid onto a 2-D image of the heart chambers overall.

Optionally, one or more of the views of FIGS. 4B-4C is shown and updated as the annuloplasty device is implanted to show fastener positions. The positions of the cutaway ends of the view of FIG. 4C (e.g., cutaway surface 406) are optionally changed during implantation to more clearly show activity at the site of a currently implanting fastener. In some embodiments, cutaway surface 406 is positioned adjacent to this site. Optionally, a depth of penetration of the valve annulus by a currently implanting fastener is indicated by showing the fastener's position at or near cutaway surface 406.

In FIG. 2F, the implantation is nearly complete. In the orientation shown, the implantation was begun along the septal wall anterior to the location of the bundle of His 60, and terminates before reaching back around to the bundle of His 60 again.

Partial cinching potentially occurs accidentally during implantation. In some embodiments, this is optionally noted by changes in the imaged environment, and/or a sudden change in measured amounts of regurgitation. In some embodiments, such instances are displayed to the physician, optionally with a warning.

In FIG. 2G, implantation is complete and the annuloplasty device 112 has been cinched, shrinking valve 57, and detached. Optionally, the procedure includes final validation checks, performed before and/or after cinching and detachment.

### Post-Implantation Validation

Returning to FIG. 1: at block 119, in some embodiments, implantation is verified and/or corrected. Some aspects of validation are optionally performed during implantation itself, for example, as already described in connection with the activities of block 120.

Optionally, one or more fasteners 122 is misplaced and/or dislodged during a procedure such that it requires post-implantation removal. In some embodiments, post-implantation imaging is used to identify such instances, and/or to guide a retrieval tool to retrieve fastener 122.

In some embodiments, there is a period of post-implantation adaptation (e.g., of about 30 minutes), during which tension and/or compression induced on the valve annulus by the implant results in initial valve remodeling. In some embodiments, measurements (e.g., by Doppler ultrasound and/or of saline injection retrograde transport) are performed to confirm that results anticipated for regurgitation reduction have actually been obtained. In some embodiments, electrophysiological activity of vulnerable structures is measured, to confirm that no inadvertent electrophysiological block has developed during the period of adaptation (e.g., due to pressure exerted by the annuloplasty device on nervous tissue). In some embodiments, rearrangements of positions of fasteners 122 after valve remodeling are imaged, for example, to confirm that they have not been brought into closer-than-intended proximity to vulnerable structures such as the coronary artery, AV node, and/or bundle of His. Once valve remodeling has stabilized (e.g., changing shown in valve images has slowed and/or stopped), the implantation procedure is optionally deemed complete, equipment is removed from the patient, and the patient is released.

### Mitral Valve Annuloplasty

Reference is now made to FIG. 3A, which schematically illustrates implantation of an annuloplasty device 112 for treatment of regurgitation in a mitral valve 47, according to some embodiments of the present disclosure. Reference is also made to FIG. 3B, which is a schematic flowchart of a method of guiding and monitoring implantation of a mitral heart valve annuloplasty device 112, according to some embodiments of the present disclosure.

Shown in FIG. 3B is an almost fully deployed and attached annuloplasty device 112, comprising sleeve 121, cinch cord 125, fasteners 122, and optional radiopaque markers 123. A control member 124 is shown still connected to fastener 122A, as faster 122A is being attached (e.g., screwed in) to the annulus of valve 47 (this process is described, for example, in relation to FIG. 2E). Also shown is delivery catheter 112A, from which annuloplasty device 112 is being deployed and attached. A multi-electrode catheter 102 is also shown; both devices have been advanced into left atrium 49 of heart 50 from the right atrium 51 via a transseptal access (e.g., across the interatrial septum via the foramen ovale 43).

Also illustrated in FIG. 3B are left atrial appendage 46, and the roots of pulmonary veins 48. Left ventricle 42 is illustrated below mitral valve 47, including papillary muscles 45 of the left ventricle 42, chordae 44, and the aortic root 54.

The blocks 310, 312, 314, 316, 318, 320, 322 of FIG. 3A correspond generally to blocks 110, 102, 114, 116, 118, 120, 119 of FIG. 1, with the substitution of the left atrium 49 for the right atrium 51, of left ventricle 42 for right ventricle 55, and of the mitral valve 47 for the tricuspid valve 57.

Structures to avoid damaging and/or anchoring to directly in mitral valve annuloplasty continue to include the AV node 60A, bundle of His 60, coronary artery 59 (the left branch, in particular), and valve leaflets. Anchoring and/or damage to walls and other non-valvular structures of the left atrium 49 and left ventricle 42 is also avoided.

### Valve Feature Tagging

In some embodiments, valve and peri-valvular features including the valve leaflets, valve annulus (fibrous tissue of the valve annulus, bounded internally by the “hinge” of the valve, and externally by myocardial tissue), and surrounding cardiac tissue are detected, distinguished, and tagged for presentation as an image.

In some embodiments, presentation of features as an image comprises presentation as a 3-D (or 2-D projected 3-D) image, with particular structures distinguishably tagged, for example, by a marker; and/or by differences in color, brightness, saturation, transparency, texture, or another visual characteristic.

Some examples of how different valve and peri-valvular structural elements are distinguished from each other are discussed below. Others, for example, detection of vulnerable structures, are discussed, for example, in relation to FIG. 1.

### Valve Annulus Tagging

Reference is now made to FIG. 7A, which schematically illustrate a method of identifying valve hinge locations, according to some embodiments of the present disclosure.

At block 702, in some embodiments, location voltage measurements 701 (corresponding, for example, to the measurements used to produce the position map of block 110) are taken as an input, and the position map produced. The position map potentially already at least partially identifies the hinge of a valve, based on the location of the “cliff” edge which marks the transition from the approximately valve-aperture transverse surface of the valve annulus to the approximately valve-aperture perpendicular surface of the valve leaflets. Additionally or alternatively, manual or automatic identification of the “cliff” edge is facilitated by restricting the search for a suitable geometrical feature corresponding to a valve hinge to a region along the atrioventricular axis identified as plausible based on electrophysiological measurement of a waveform characteristic of an annular ring position. For purposes of automatic identification, the hinge is optionally identified as comprising a circumferentially extending ring (optionally broken, e.g., at the leaflet boundaries), whereat the elevation angle of surface orientation relative to the plane of the valve annulus is changing in a manner characteristic of the hinge. For example, the change rate is above a threshold, and/or the change rate is fastest at some particular radial distance from the valve annulus.

Block 704 represents intracardiac electrograms, corresponding, for example, to measurements of block 116 of FIG. 1 which map endogenous electrophysiology. It is described herein (e.g., in relation to block 116 of FIG. 1) that electrophysiological waveforms with different characteristics (e.g., relative amplitudes of components) are optionally used to help identify locations such as the AV node and/or bundle of His; and/or to identify locations along the atrioventricular axis. In particular, intracardiac locations having electrophysiological waveforms mid-way between the P-wave dominated (atrial) and QRS-complex dominated (ventricular) are optionally identified as being at the level of the valve annulus.

Block 706, in some embodiments, corresponds to electrical measurements made in spatial coordination with (e.g., at the same places as) the intracardiac electrogram measurements of block 704. Optionally, block 706 includes measurements of local dielectric properties (as indicated in components of electrical impedance affected by nearby tissue, and particularly by contacts with nearby tissue). In particular, the valve annulus ring comprises connective tissue of a composition with impedance properties that are potentially distinct from nearby contractile muscular tissue (e.g., atrial muscle outside the annulus ring), and also potentially distinct (e.g., due to thickness, composition, and/or movement patterns) from impedance measurements made while in contact with the valve leaflets. Distinguishing base on movement patterns is also described in relation to FIGS. 7B and/or 8, herein.

Additionally or alternatively, block 706 includes location voltage measurements (e.g., of externally induced electrical fields), for example of the type optionally used as the location voltage measurements of block 701. Location voltage measurements are voltage measurements indicative of locations; for example, measurements providing a basis for a process of computational reconstruction, e.g., as described in WO 2019/034944.

The same electrode(s) are optionally used for any of the electrical measurements of block 706 (e.g., location voltage measurements and/or dielectric measurements). Simultaneous measurements, in some embodiments, are at least partially separable from each other within a single time series of recorded measurements, for example based on differences in frequency, and/or by using differential analysis techniques in comparison to measurements from other locations. For example, components of measurements due to the externally induced electrical fields can be distinguished based on frequency of the induced electrical fields. Measurement influences due to local dielectric properties are observable at other frequencies, and moreover become particularly pronounced upon making contact with local tissue; to the extent that a sudden large change in impedance is itself potentially indicative of tissue contact using the measuring electrode. The dielectric properties of the relative fibrous valve annulus are different from those of the valve leaflets (on one side) and the cardiac tissue (on the other), leading to a difference in dielectric property-attributable measurement signals upon contact with each of these different tissue types. A “local” component of nearby measurements which are sensitive to both local and diffuse signal sources can in some cases be isolated by signal subtraction (e.g., when one of the measurements is in a relatively quiet local environment), or by another differential analysis technique.

In some embodiments, positions and/or characteristics of the location measurements of block 706 are used to assign positions to the intracardiac electrogram measurements of block 704 within the position map of block 702.

At block 708, positions of locations tagged as “hinge” (valve annulus) positions are output, based on processing of inputs to block 709 from blocks 702, 704, and 706. In some embodiments, measurements made from positions which are generally at the level of the valve annulus are validated by intracardiac electrogram measurements which sufficiently correspond to measurements expected from the level of the valve annulus along an atrioventricular axis (e.g., as described in relation to block 116 of FIG. 1). Local dielectric properties due to tissue characteristics (thickness, tissue type, and/or movement) provide a further distinguishing characteristic (e.g., distinguishing valve annulus from valve leaflets and/or myocardial tissue). From detailed structural (shape) measurements of the heart lumen, in some embodiments, there may be distinguished the position of the edge of a “cliff” which drops suddenly into the ventricle from the atrium, and this also is a marker of the valve annulus position. The information from any one or more of these types of measurements optionally is used (including used jointly) to create a tissue-type tagged model of the valve annulus.

In some embodiments, structures of the valve are in part differentiated using analysis of the temporal frequencies of motion of different structural elements of the valve, for example as described in relation to FIG. 7B.

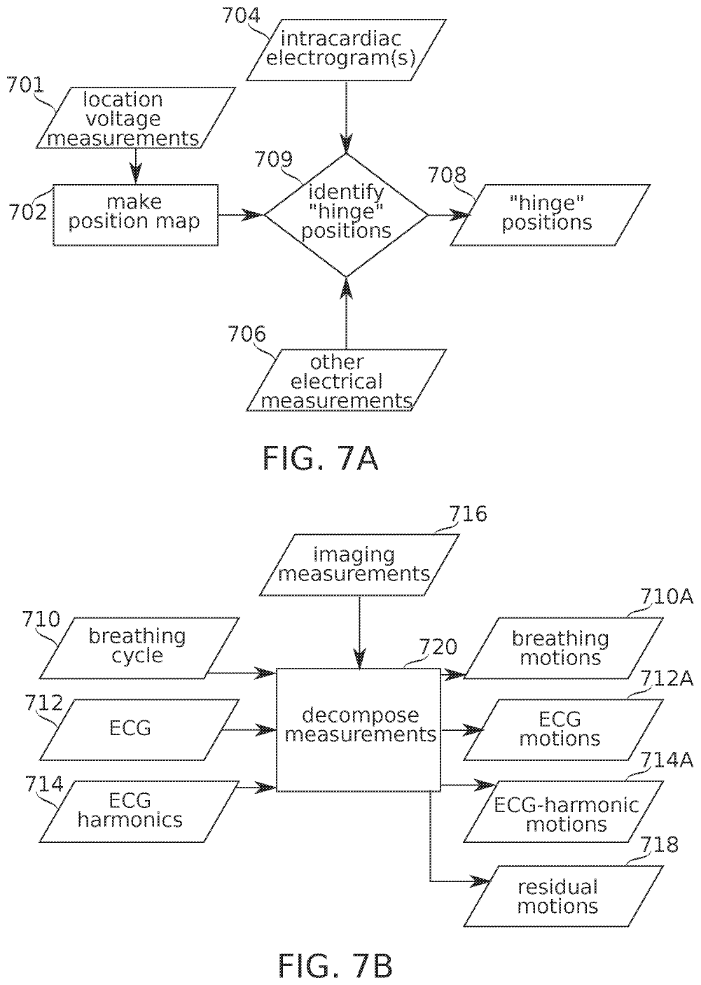

Optionally, locations of valve hinge vs. valve leaflets are distinguished at least partially on the basis of impedance measurements indicating wall contact (for example, as described in relation to FIG. 8); for example, contacts of a relatively stationary probe with valve leaflets may tend to be intermittent, compared to contacts with the valve hinge (annulus).

### Valve Leaflet Tagging

Reference is now made to FIG. 7B, which schematically illustrates a method of using time-frequency decomposition to distinguish components of heart structure as belonging to different structures, according to some embodiments of the present disclosure. Reference is also made to FIG. 5B, which schematically represents time traces of respiration (trace 501), and body surface ECG (trace 502).

As imaging measurements (e.g., based on electrical impedance tomography, or another imaging method) are made of the region of a heart valve over the course of several heart and respiratory cycles, different parts of the valve move in different ways. For example, the valve annulus (e.g., of the tricuspid valve) experiences longitudinal motion in response to contractile actions of surrounding cardiac tissue, and may experience slower motions as a result of lung and diaphragm movements. Valve leaflets may also have movements at higher frequencies (e.g., harmonics of the heartbeat frequency): for example a brief partial opening at a phase other than the valve's main opening. Where imaging data is obtained, e.g., using internally placed electrodes positioned at a vantage point substantially along an axis of motions of the valve (e.g., an atrioventricular axis, in the case of the tricuspid valve or the mitral valve), the valve may be considered as laid out approximately across an X-Y axis-defined plane, and its motions may be understood as happening approximately toward and away from the vantage point of the images along a Z axis (e.g., atrioventricular axis). This motion can be understood as “painting” different parts of the X-Y axis with different cyclic motion-pattern “colors”, carrying information that distinguishes, e.g., valve leaflets from the valve annulus itself.

In some embodiments, different cyclic movements of tissues belonging to different structural parts of the valve are differentially labeled by what is known in the field of image feature detection as “blob detection”. Areas “colored” with high power in harmonics of the heartbeat frequency are labeled as valve leaflets; other areas are not. In some embodiments (for example as shown in FIG. 7B), spectral power (amplitude variation of the measured waveform over time) is decomposed to (attributed to) respiratory frequency, cardiac cycle frequency, higher cardiac cycle frequency harmonics, and residual processes. The resulting distribution of spectral power is categorized, e.g., using a Hessian operator-based method of blob detection, or another method, for example, a machine learning-based method that uses established associations of feature locations to measurements at those locations as input, and from this input learns which measurements indicate which features.

In some embodiments, image feature detection comprises receiving measurements 710, 712, 714 of the breathing cycle (e.g., trace 501 of FIG. 5B), body surface ECG (cardiac cycle, trace 502), and higher harmonics of the body surface ECG (respectively) to a decomposition module 716, which uses measurements 710, 712, 714 to decompose and tag the time- and space-indexed imaging measurements of block 716; tagging the spatial locations according to the magnitude of their various cyclic motions—breathing 710A, cardiac cycle 712A, higher cardiac cycle harmonics 714A, and residuals 718.

Valve leaflets may, additionally or alternatively, be modeled parametrically using constraints established by impedance measurements obtained over time. For example, each valve leaflet is modeled as tissue anchored on one side to the valve annulus (with a certain parametrically defined circumference), over a certain parametrically defined distance of the circumference, and with certain parametrically-defined shapes of its commissural sides. The effects of any particular set of parametrically defined leaflets on impedance measurements can be determined by using impedance properties of the tissue, and well-known equations of how impedance changes modify electrical field distribution. The impedance measurements, accordingly, constrain the parametrically defined configuration of valve leaflets that is actually present. This configuration can be found, for example, by an iterative process of valve parameter adjustment that leads to a closer fit between actual measurements (and in particular, in some embodiments, the component of actual measurement attributable to valve motions) and predicted measurements.

### Tissue Wall Contacts

Reference is now made to FIG. 8, which schematically represents detection of wall contacts, according to some embodiments of the present disclosure.

In some embodiments, locations in contact with tissue surfaces are distinguished using impedance measurements. Block 810 represents position measurements made (e.g., using impedance measurements) by a probe (e.g., an electrode probe) moving around within the heart chambers, and block 812 represents measurements of impedance sensed by an electrode of the probe between itself and an external electrode. As the electrode approaches and then contacts surface of the heart chambers, impedance rises. Impedance rises corresponding to the characteristics of lumenal wall surface contacts are detected at block 820, the output of which is used to tag positions as wall contacting (block 816) or lumenal (non-wall contacting) (block 818).

In some embodiments, tissue wall contacts in the peri-valvular region which are not known (e.g., by other segmentation tests such as those of FIGS. 7A-7B) to be of another structural type such as the valve annulus or valve leaflet are assigned as cardiac muscle tissue.

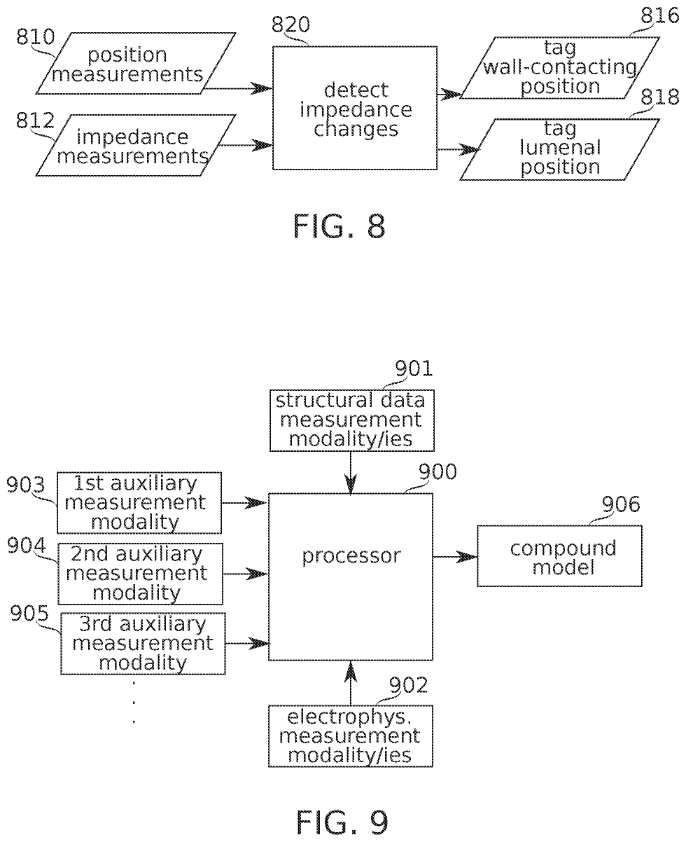

Reference is now made to FIG. 9, which is schematic diagram of a system for monitoring and/or guiding annuloplasty device implantation, according to some embodiments of the present disclosure.

Computer processor 900 is configured to execute computer code instructions for integrating data acquired from data measuring and/or processing subsystems supporting a plurality of measurement modalities (for example, measurement modalities supported by data measuring and/or processing subsystems 901, 902, 903, 904, and/or 905) into a compound model 906 of a heart. In some embodiments, compound model 905 models at least a heart valve and its vicinity. Integration of data from subsystems 901-905 comprises, for example, use of simultaneity of measurements obtained using different subsystems 901-905, and/or another method of establishing coordination between different measurement modalities, for example as discussed in relation to FIG. 1. While any of the measuring and/or processing subsystems 901-905 is optional, there are in general at least two of them in operation to provide inputs to processor 900. In some embodiments, the system of FIG. 9 includes a display, through which the processor displays output.

Compound models 906 and aspects of their determination are discussed in relation to several different embodiments herein; for example, in relation to FIGS. 1, 3A, and/or 7A-8. Other example descriptions follow, each an example of a relevant plurality of subsystems indicated:

Systems configured to map a vascular lumen extending alongside a body cavity. In some embodiments, first and second probes moving within each respective lumen comprise separate modalities of position measurement (two instances of subsystem 901). The compound model 906 optionally comprises, for example, a combined spatial representation of the two lumens, and/or a model of distances between first lumen locations and second lumen locations. Optionally, first lumen locations and distances to the second lumen which are “too small” (e.g., below a threshold) are considered “dangerous” for certain operations such as implantation/attachment, and indications are produced to help an operator avoid performing those operations in the dangerous areas.

Systems configured to locate a heart valve annulus along an atrioventricular axis. In some embodiments, electrophysiological signals are measured from probe positions extending between an atrial side and a ventricular side of a heart valve annulus (subsystem 902), while probe positions are also measured (subsystem 901). Position-dependent characteristics of the electrophysiological signals are used to identify which positions are apparently within the region of the heart valve annulus.

Systems configured to detect valve leaflets. In some embodiments, measurements of electrical signals indicative of impedance changes from one or more electrodes located near a cardiac valve (subsystem 903, comprising sensor and processing to perform time-series intracardiac impedance measurements) are processed to determine the presence and/or details of electrical signal changes characteristics of proximity to a leaflet of the cardiac valve. Position information (e.g., obtained using a structural data measurement modality 901) is optionally used to structure a spatial model of the position measurements.

Systems configured to identify hinge boundaries between a heart valve annulus and heart valve leaflets. In some embodiments, inputs are received which measure a time course of changes in an electrical signal indicative of impedance changes due to motion of tissue; more particularly, in some embodiments, at a plurality of probe positions in proximity to one or both of the heart valve annulus and the heart valve leaflets. This corresponds to an example of subsystem 903, comprising an electrode sensor and processing to perform time-series intracardiac impedance measurements. Using position information of the probe (e.g., obtained using a structural data measurement modality 901), relative spatial locations of the probe positions within the heart are determined to be valve hinge positions, based on being at locations between valve annulus locations (having one type of motion signal recorded using subsystem 903) and valve leaflet locations (having another type of motion signal recorded using subsystem 903).

Systems configured to locate a structure of the electrical conduction system of the heart. In some embodiments, measurements of intracardiac electrophysiological signal waveforms made are at intracardial probe positions (subsystem 902), and spatial locations of the probe determined separately (subsystem 901). The electrophysiological waveforms and spatial information are processed together to identify at least one of the spatial locations as being at the position of the electrical conduction system structure.

In some embodiments, systems are configured to use a compound model (e.g., as may be generated by any of the above-described systems) to generate a specification of implantation positions within a heart posing a risk; for example, risk of damage to a right coronary artery, a bundle of His, or another functionally crucial region of the heart. Positioning of a portion of an implantable device (e.g., a fastener of an annuloplasty device) may be tracked according to one or more of subsystems 901, 902, 903, and compared with the compound model to reach targeted structures, and/or to help reduce a risk of causing damage to identified structures.

In some embodiments, systems are configured more particularly to monitor the implantation of a fastener for an annuloplasty device into a valve annulus. In some embodiments, measurements of an electrical signal indicative of impedance using the fastener as an electrode are received while the fastener is being brought to an implantation position (instance of a subsystem 903). Again, in some embodiments, locations of other structures have been previously mapped, for example according to operations outlined with respect to any of the above-described systems.

In some embodiments, a systems according to FIG. 9, or another system comprising processor and suitably memory-stored instruction, is configured to implement one or more feature-finding algorithms, for example:

Algorithms to automatically locate a hinge of a heart valve annulus.

Algorithms to define a nominally safe pathway extending along and between the locations of two circumferentially extending portions of the heart comprising a hinge portion of the heart valve, and a portion of a coronary artery.

In some embodiments, subsystem 901 comprises devices and/or software implementing a measurement modality which provides data supporting production of a 3-D structural image of the modeled portion of the heart. Subsystem 901 itself may comprise, for example, computer code configured to receive electrical field measurements (e.g., from a plurality of electrodes of a probe inserted into the heart) and convert them into positions associated with the measurements. Optionally, subsystem 901 also comprises electrical field measurement hardware such as an electrode probe (e.g., having a plurality of electrodes at a known relative distance), a measurement controller functionally configured to measure voltages, currents, and/or impedances from electrodes of the electrode probe, a catheter over which the electrode probe is delivered to a lumen of the heart, analog-to-digital conversion circuitry, a memory store for recorded measurements, and/or a digital communication link for transmitting measurements made by subsystem 901 to computer processor 900.

Additionally or alternatively, in some embodiments, subsystem 901 comprises 3-D ultrasound equipment, configured to measure, record, and transmit 3-D ultrasound measurements and/or images to computer processor 900.

In some embodiments, subsystem 902 comprises devices and/or software implementing a measurement modality which measures electrophysiological signals produced by endogenous and/or stimulus-evoked activity of cardiac tissue. In some embodiments, subsystem 902 includes, for example, one or more electrodes, a probe carrying the one or more electrodes, a measurement device for voltage, current and/or impedance, analog-to-digital conversion circuitry, a memory store for recorded signals, and/or a digital communication link for transmitting measurements made by subsystem 902 to computer processor 900.

In some embodiments, subsystems 903-905 (there may be one, two, three, or more such subsystems; three are shown for purposes of description) comprise devices and/or software (“equipment”) implementing other measurement modalities, and in functional communication with computer processor 900. Examples include:

Body surface ECG equipment.

Respiratory cycle motion measurement.

X-ray imaging equipment.

Echocardiography equipment; configured for example to measure valve shapes and/or Doppler signals due to regurgitation.

Equipment to inject saline and measure retrograde saline transport (e.g., as a measurement of regurgitation).

Catheter and recording and/or generating equipment configured to insert a wire electrode to a blood vessel (for example, a coronary artery), and record and/or generate an electrical signal therefrom.

Catheter and recording and/or generating equipment configured to insert an electrode catheter (e.g., a catheter comprising a distal probe with a plurality of electrodes along its length) to a blood vessel (for example, a coronary artery), and record electrical signals therefrom which are indicative, under a suitable voltage-to-spatial transformation, of the spatial positioning of the blood vessel.

### Coronary Artery Proximity and Penetration Detection

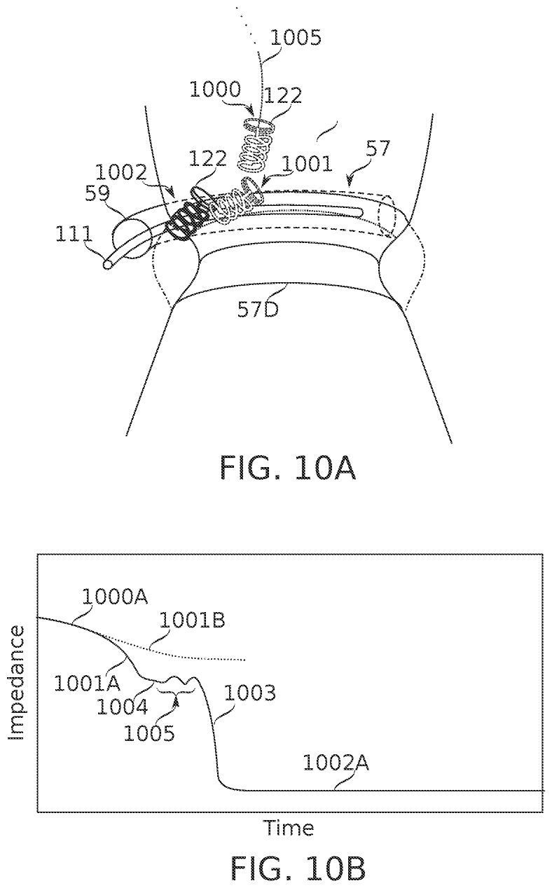

Reference is now made to FIG. 10A, which schematically represents coronary artery proximity and penetration by a device fastener 122, according to some embodiments of the present disclosure. Reference is also made to Figure JOB, which schematically represents features of coupling measurements potentially useful to detect changes of coronary artery proximity and penetration by a fastener, according to some embodiments of the present disclosure. Figure JOB illustrates idealized measurements of impedance over time, with, e.g., measurement noise suppressed to assist in illustrating main features.

In some embodiments of the present disclosure, a coronary artery (e.g., in the right atrium, the right coronary artery) is at potential risk for damage during a valve annuloplasty, or optionally another structural procedure performed in heart lumen regions underlying the coronary artery. FIGS. 10A-10B illustrate methods of avoiding and/or detecting coronary artery damage due to contact and/or penetration by a fastener 122. Fastener 122 may be, for example, a fastener of a valve annuloplasty device, or another implantable device.

FIG. 10A shows fastener 122 in three positions 1000, 1001, and 1002 corresponding to times and relative impedances 1000A, 1001A, and 1002A of Figure JOB, respectively. Fastener 122 is connected to an electrical sensing and/or driving system, for example via wire 1005, which for clarity of illustration is shown only in a distal portion thereof for the case of position 1000. Also for clarity of illustration, the device being fastened with fastener 122 is omitted from the drawing. Its relationship with fastener 122 may be, for example, as illustrated and/or discussed in relation to FIG. 3B, or another figure herein.

At position 1000, fastener 122 approaches the vicinity of catheter wire 111. Catheter wire 111 has been previously inserted into coronary artery 59; for example as described in relation to FIG. 2B. In some embodiments, for example, catheter wire 111 is a catheter probe, having on it electrodes. At least one of catheter wire 111 and fastener 122 is driven to generate a small (e.g., <1 mA) electrical current (e.g., an AC electrical current at about 10-40 kHz); similarly, at least one of the two is configured for use in sensing one or more parameters of electrical coupling between the two (e.g., impedance). The trace in the region of time/impedance point 1000A represents this coupling in the form of impedance.

At position 1001, fastener 122 approaches coronary artery 59 more closely, and the coupling increases. This may be measured, for example as a drop in impedance to the level of the region of time/impedance point 1001A. There may also be impedance change effects as a function of orientation, depending on the geometry and electrical conductivity characteristics of fastener 122.

This allows using impedance between fastener 122 and catheter wire 111 in a method of judging relative distance between the fastener and the coronary artery.

One way that relative values can be used in judgement is by noticing the difference between impedances achieved upon approaches two different areas of valve annulus 57D. Approach to a “landing point” (fastening position) further from catheter wire 111 (and the coronary artery it occupies) will potentially not increase coupling as much and/or as quickly as an approach to a landing point which is closer, and correspondingly at greater risk for introducing a complication of coronary artery penetration. Region 1001B (Figure JOB) shows such a reduced change in coupling as a reduced change in impedance. Thus, different candidate fastening positions can be compared, and an apparently less-risk position selected.

A plateau region (plateau 1004, for example) may indicate that contact with tissue resistant to further movement has been made, particularly if the plateau persists with minimal reduction in response to attempts to advance fastener 122 further.

Region 1005 indicates semi-cyclical changes in impedance which may, in some embodiments, appear as a helical fastener 122 is driven into tissue (e.g., as a tip of fastener 122 rotates toward and away from catheter wire 111. Other fastener geometries may be associated with different characteristic “penetration” profiles of a coupling measurement such as impedance.

Through region 1003, a penetration event is occurring, representing a rapid increase in coupling (drop in impedance) as a tip of fastener 122 intrudes into coronary artery 59. The impedance drop may reflect the lower resistivity of blood in comparison to the solid tissue of the valve annulus. At region 1002A, a new, lower plateau of impedance is reached.

This provides a potentially immediate indication to an operator such as an implanting physician that a complication involving the coronary artery has likely occurred. This allows quickly halting the advance of a fastener before more damage occurs. There can also be taken rapid remedial steps to repair the damage which has occurred, e.g., by cauterization, or switching to another mitigation procedure.

It is provided, in some embodiments, that an alarm (visual, haptic, and/or auditory; optionally, an intrusive alarm—loud and/or garish, for example) is used to warn an operator of the likelihood of a penetration event, detected by a sudden impedance drop. The physician optionally sets the threshold for the alarm in terms of coupling change (e.g., impedance drop) magnitude and speed. Optionally, one or more presets may be provided.

Impedance changes can occur for different regions during a procedure, and it is a potential advantage to reduce the production of false positive alarms.

Optionally, in some embodiments, production of an alarm upon detection of one or more of the coupling/impedance waveform characteristics just described is gated by at least one additional criterion that helps increase the likelihood that the event detected is really a penetration event. In some embodiments, the additional criterion is provided by measuring the position of fastener 122 along an atrial-ventricular axis, for example as described in relation to FIGS. 2E and/or 7A. Accordingly, only when the position is determined to be within the region of the valve annulus will a determination of a likely penetration event be made and/or converted to an alarm to the operators.

It should be noted that coupling between catheter wire 111 and fastener 122 may be measured in another way, for example using vibrations (e.g., as described in relation to FIG. 2E), or another electromagnetic measurement method. The monitoring of impedance as a measure of coupling (described as an example here) has been found to be notably sensitive to penetration events.

It is noted that relative position (e.g., distance and/or direction) between a first probe and catheter wire 111 within a coronary artery may be measured while tracking positions of the first probe by a separate indication—for example, measurements of voltages within a plurality of crossing electrical fields. This can be used to generate a map of regions close enough to the coronary artery to raise potential concern; for example, concern for risk of damage during device implantation. In some embodiments, proximity to the coronary artery for another probe (e.g., the fastener 122 itself), can be tracked using the same separate indication of position. Proximity to the coronary artery can then be determined from the map. It is noted that the pre-mapped proximity determinations can be used without direct coupling (e.g., current and/or voltage measurement) between the fastener 122 and catheter wire 111. Furthermore, once mapping is performed, and catheter wire 111 can be removed from coronary artery 59.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-5. (canceled)

6. A system configured to locate a heart valve annulus along an atrioventricular axis, the system comprising:
a processor, memory storing instructions, and display;
wherein the processor is configured to receive intracardiac electrophysiological signal waveforms measured from:
probe positions extending between an atrial side and a ventricular side of a heart valve annulus,
including, for each side, a respective plurality of probe positions;
wherein the processor operates according to the instructions to:
determine relative spatial locations of the probe positions within the heart,
identify, based on the signal waveform measurements, a position and orientation of a region between the atrial side and the ventricular side, the region being positioned at the heart valve annulus along the atrioventricular axis, and oriented to include opposite circumferential sides of the heart valve annulus, and
produce a model of the heart.

7. The system of claim 6, wherein the spatial locations correspond to locations within a 3-D model of the heart.

8. The system of claim 7, wherein the processor further operates to present on the display the 3-D model of the heart marked with the identified spatial locations at the position of the heart valve annulus along the atrioventricular axis.

9. The system of claim 6, wherein the processor operates to identify at least one of the spatial locations as being at the position of an atrium along the atrioventricular axis, based on the signal waveform measured at the at least one of the spatial locations.

10. The system of claim 9, wherein the processor operates to present on the display a 3-D model of the heart marked with the identified at least one of the spatial locations at the position of the atrium along the atrioventricular axis.

11. The system of claim 6, wherein the processor operates to identify at least one of the spatial locations as being at the position of a ventricle along the atrioventricular axis, based on the signal waveform measured at the at least one of the spatial locations.

12. The system of claim 11, wherein the processor operates to present on the display a 3-D model of the heart marked with the identified at least one of the spatial locations at the position of the ventricle along the atrioventricular axis.

13. The system of claim 6, wherein the processor identifies position along the atrioventricular axis by identifying relative amplitudes of an atrially-generated electrophysiological signal, and a ventricularly-generated electrophysiological signal.

14. The system of claim 13, wherein the atrially generated signal comprises a P wave of an electrocardiogram.

15. The system of claim 13, wherein the ventricularly generated signal comprises a QRS complex of an electrocardiogram.

16. The system of claim 6, wherein the processor identifies position along the atrioventricular axis by interpolating between atrial-side and ventricular side measurements of the electrophysiological signal waveforms to identify one or more intermediate positions at the heart valve annulus.

17. The system of claim 6, wherein the processor identifies position along the atrioventricular axis by identifying a planar region intersecting an entire circumference of the valve annulus.

18. The system of claim 6, wherein the processor identifies position along the atrioventricular axis by identifying a non-planar region intersecting an entire circumference of the valve annulus.

19. The system of claim 18, wherein the non-planar region is saddle-shaped due to a geometric deformity of the valve annulus.

20-52. (canceled)
